(19) United States
(12) Patent Application Publication
(10) Pub. No.: US 2024/0068023 A1
(43) Pub. Date: Feb. 29, 2024

(54) METHODS TO PREDICT EFFECTS OF PERIOPERATIVE ADMINISTRATION OF METHADONE TO OPTIMIZE PAIN RELIEF AND AVOID ADVERSE EFFECTS

(71) Applicant: The Trustees of Indiana University, Bloomington, IN (US)

(72) Inventor: Senthilkumar Sadhasivam, Bloomington, IN (US)

(21) Appl. No.: 18/261,451

(22) PCT Filed: Jan. 12, 2022
(86) PCT No.: PCT/US2022/012088

§ 371 Date: Jul. 13, 2023

Related U.S. Application Data
(60) Provisional application No. 63/211,129, filed on Jun. 16, 2021.
(60) Provisional application No. 63/137,208, filed on Jan. 14, 2021.

(51) Int. Cl. C12Q 1/6869; A61K 31/137; A61K 45/06; G16B 20/20; G16H 20/10

(52) U.S. Cl. CPC C12Q 1/6869; A61K 31/137; A61K 45/06; G16B 20/20; G16H 20/10; C12Q 2600/106; C12Q 2600/156

(57) ABSTRACT

A method for controlling pain in a patient in need thereof includes administering to the patient multiple low doses of methadone, wherein each subsequent dose of methadone is administered within an effective period of time from a previously administered dose of methadone, and wherein the patient has no respiratory depression and no QT prolongation. A method for providing personalized analgesic therapy to a surgical patient includes directing preoperative genotyping of a patient to determine what allele is present at a gene locus to obtain a patient-specific genetic data set, wherein the gene locus is one or more of a locus that encodes an enzyme or protein or transporter or receptor associated with methadone metabolism and responses; producing a prediction of the patient's response to perioperative methadone administration based on the data set; and making a determination whether to administer methadone and a precision perioperative dosing regimen.

### CROSS-REFERENCE TO RELATED APPLICATIONS

This application claims priority to U.S. Provisional Patent Application No. 63/137,208, which was filed Jan. 14, 2021, and to U.S. Provisional Patent Application No. 63/211,129, which was filed Jun. 16, 2021. The contents of both applications are incorporated by reference herein.

### STATEMENT ON FEDERALLY SPONSORED RESEARCH OR DEVELOPMENT

This invention was made with government support under HD089458 awarded by the National Institutes of Health (NIH). The government has certain rights in the invention.

### BACKGROUND

Intraoperative methadone has been used for surgical pain relief for a few decades, and it is being increasingly used in children. Methadone is commonly used as a racemic mixture, and exhibits a significant variability in response in terms of analgesic efficacy and adverse events. The analgesic efficacy of intraoperative methadone has been reported in both adults and children. Costly adverse effects including prolonged hospitalization due to common adverse effects such as vomiting, sedation, as well as serious adverse effects including life-threatening respiratory depression and deaths have been reported with the use of methadone. Methadone has significant inter-individual variations in response. The optimal precision dosing strategy and personalized use for optimal clinical and healthcare economic outcomes largely remain inconclusive in children and adults receiving methadone for surgical pain relief intraoperatively and postoperatively. Though most of scientific studies have used 0.1 mg/kg to 0.3 mg/kg of an intraoperative dose of methadone, the safety of such a dose has not been established. In adolescents undergoing posterior spinal fusion, intraoperative methadone resulted in only minimal reduction in total opioid use without sustained analgesia. Despite the long elimination half-life of methadone, rapid redistribution after intravenous administration could result in significant decrease in the analgesic duration, especially when small doses are used. Thus, a single dose in adolescents can result in suboptimal analgesia irrespective of the dose administered with the plasma concentration dropping below the minimum effective analgesic concentration within an hour resulting in a recommendation for additional doses of methadone or infusion following intraoperative methadone to ensure sustained analgesia. During the last decade, reinvigoration of the use of intraoperative methadone has been slowed due to fear of serious adverse effects including respiratory depression and inconsistent analgesia in adults and children. More personalized, effective, and safe methadone regimens with precision dosing strategies for surgical procedures are needed for reduced methadone-related adverse effects, and for wide adoption to obtain unique benefits including improved acute surgical analgesia, reduced need for short-acting opioids, reduced risk for opioid dependence and addiction, and lower risk for chronic postsurgical pain.

### SUMMARY

According to an embodiment, a method for controlling pain in a patient in need thereof includes administering to the patient multiple low doses of methadone, wherein each subsequent dose of methadone is administered within an effective period of time from a previously administered dose of methadone, and wherein the patient has no respiratory depression and no QT interval prolongation in electrocardiogram.

In some embodiments, the methadone is administered as part of a multimodal analgesia therapy.

In some embodiments, the method further includes administering to the patient at least one additional active ingredient.

In some embodiments, the administration is intravenous or oral.

In some embodiments, the effective period of time is about 12 hours.

In some embodiments, the pain is controlled for about 12 hours.

In some embodiments, the pain is associated with a surgery performed or to be performed on the patient.

In some embodiments, the surgery is an orthopedic surgery.

In some embodiments, the methadone is administered to the patient preoperatively.

In some embodiments, the methadone is administered to the patient intraoperatively.

In some embodiments, the methadone is administered to the patient postoperatively.

In some embodiments, the patient has a lower incidence of chronic pain after the surgery relative to an incidence of chronic pain in a population that does not receive multiple small doses of methadone.

In some embodiments, the patient has an improved postoperative functional disability index score relative to a postoperative functional disability index score in a population that does not receive multiple small doses of methadone.

In some embodiments, the patient has a reduced postoperative opioid use with reduced risk for opioid-related respiratory depression, dependence and addiction relative to a population that does not receive multiple small doses of methadone.

In some embodiments, the concentration of postoperative alpha-1 acid glycoprotein (AAG) in the patient increases in response to the surgery.

In some embodiments, a concentration of postoperative AAG in the patient changes after surgery and influences a response and a pain relief profile of the methadone.

In some embodiments, the concentration of postoperative AAG is inversely related to the concentration of methadone and pain relief.

In some embodiments, each dose of methadone is at a dose of 0.01 mg/kg of weight of the patient to 1 mg/kg of weight of the patient.

In some embodiments, each dose of methadone is at a dose of 0.01 mg/kg of weight of the patient to 0.3 mg/kg of weight of the patient with a maximum dose of 15 mg per dose.

In some embodiments, each dose of methadone is at a dose of 0.05 mg/kg of weight of the patient to 0.15 mg/kg of weight of the patient.

In some embodiments, each dose of methadone is at a dose of 0.1 mg/kg of weight of the patient.

In some embodiments, the multiple low doses of methadone comprise 3 or 4 low doses of methadone.

In some embodiments, the multiple low doses of methadone intraoperative and postoperatively reduce the risk of respiratory depression while improving pain relief at safe and non-toxic blood methadone concentrations.

In some embodiments, increasing and varying AAG concentrations contribute to inter-patient variations in methadone's pharmacokinetics, clinical safety, and analgesic efficacy.

In some embodiments, CYP2B6 genetic variations account for huge variations in methadone's metabolism and clinical methadone dose adjustments along with AAG.

In some embodiments, CYP2B6 genetic variations account for huge variations in methadone-induced adverse effects and post-surgical pain.

In some embodiments, OPRM1 genetic variations account for huge variations in in-hospital opioid use, methadone-induced adverse effects, and length of hospital stay.

In some embodiments, ABCB1 genetic variations account for huge variations in methadone-induced PONV.

In some embodiments, FAAH genetic variations account for huge variations in in-hospital opioid use and higher risk for methadone-induced PONV and at least 1 day longer hospital stay.

In some embodiments, CYP2B6 genetic variations account for higher risk for methadone-induced PONV.

In some embodiments, POR genetic variations account for higher risk for methadone-induced PONV.

In some embodiments, CYP2B6 genetic variations account for higher risk for inadequate surgical pain relief with methadone.

In some embodiments, DRD2 genetic variations account for higher risk for methadone-induced PONV and longer hospital stay.

In some embodiments, ORM1 genetic variations coding for AAG account for significant variations in methadone's pharmacokinetics, clearance, volume of distribution resulting in variations in adverse effects and post-surgical pain relief.

In some embodiments, the patient is a human.

In some embodiments, the patient is a child or adolescent or adult.

According to another embodiment, a method for providing personalized analgesic therapy and/or precision dosing to a surgical patient to improve post-surgical outcomes includes directing preoperative genotyping of a patient in need of surgery to determine what allele is present at a gene locus to obtain a patient-specific genetic data set, wherein the gene locus is selected from the group consisting of a locus that encodes an enzyme associated with methadone metabolism, a locus that encodes AAG and combinations thereof; producing a prediction of the patient's response to perioperative methadone administration based on the data set; and making a positive or negative determination whether to administer methadone perioperatively to the patient based on the prediction. In some embodiments, the positive or negative determination comprises a positive determination and wherein the method further comprises, after said making, directing administration of methadone to the patient.

In some embodiments, the administration of methadone to the patient comprises administration of multiple low doses of methadone, wherein each subsequent dose of methadone is administered within an effective period of time from a previously administered dose of methadone.

In some embodiments, the methadone is administered as part of a multimodal analgesia therapy.

In some embodiments, the method further includes administering to the patient at least one additional active ingredient.

In some embodiments, the administration is intravenous or oral.

In some embodiments, the effective period of time is about 12 hours.

In some embodiments, the pain is controlled for about 12 hours.

In some embodiments, each dose of methadone is at a dose of 0.01 mg/kg of weight of the patient to 1 mg/kg of weight of the patient.

In some embodiments, each dose of methadone is at a dose of 0.01 mg/kg of weight of the patient to 0.3 mg/kg of weight of the patient with a maximum dose of 15 mg per dose.

In some embodiments, each dose of methadone is at a dose of 0.05 mg/kg of weight of the patient to 0.15 mg/kg of weight of the patient.

In some embodiments, each dose of methadone is at a dose of 0.1 mg/kg of weight of the patient.

In some embodiments, the multiple low doses of methadone comprise 3 to 5 low doses of methadone.

In some embodiments, the patient is a human.

In some embodiments, the patient is a child or adolescent.

In some embodiments, the genotyping comprises determining the allele present at a gene locus that encodes an enzyme selected from the group consisting of CYP2B6, CYP2D6, CYP2C19, CYP2C9 and CYP3A4, ORM1, ABCB1, FAAH, OPRM1, and combinations thereof.

In some embodiments, the genotyping comprises determining the allele present at a gene locus that encodes CYP2B6.

In some embodiments, the genotyping comprises determining the allele present at a gene locus selected from the group consisting of ORM1, ORM2, CYP2B6, and combinations thereof.

In some embodiments, the prediction comprises a prediction whether the patient, if administered methadone, will experience an acceptable analgesic effect.

In some embodiments, the prediction comprises a prediction whether the patient, if administered methadone, is likely to experience nausea and vomiting.

In some embodiments, the prediction comprises a prediction whether the patient, if administered methadone, is likely to experience excessive sedation.

In some embodiments, CYP2B6 predicts the clearance of an R-methadone and an S-methadone enantiomer in an additive gene model.

In some embodiments, the genotyping comprises determining the allele present at a gene locus that encodes intronic CYP3A4 SNP rs2246709 and said intronic CYP3A4 SNP rs2246709 is associated with decreased clearance of R-methadone and S-methadone.

In some embodiments, changing perioperative concentrations of AAG and the SNP of AAG, ORM1 SNP rs17650 independently increases the volume of distribution of an R-methadone enantiomer and an S-methadone enantiomer, thereby aiding an optimal and precision dosing of methadone.

In some embodiments, the genotyping comprises determining the allele present at a gene locus that encodes OPRM1, wherein the prediction is that a patient with an OPRM1 rs1799971 GG or GA genotype is will require 20% higher average in-hospital opioid use than AA genotype, and a patient with AA will have a higher incidence of PONV and a longer hospital stay relative to a patient without AA.

In some embodiments, a patient with a variant in ABCB1 is determined to have significantly higher risk for PONV than a patient without ABCB1.

In some embodiments, the variant is selected from the group consisting of rs1128503 and rs1045642.

In some embodiments, a patient with a homologous/heterologous variant of FAAH SNPs is expected to have 15-20% lower in-hospital opioid use compared to a patient with wild-type.

In some embodiments, the variant is selected from the group consisting of rs3766246 and rs4141964.

In some embodiments, a patient with a wild type variant of FAAH rs3766246 is expected to have a higher incidence of PONV and one-day longer hospital stay after surgery than a patient without the wild type variant of FAAH.

In some embodiments, the genotyping comprises determining the allele present at a gene locus that encodes OPRM1, and wherein a patient with POR rs1057868 TT or CT genotype is expected to have higher incidence of PONV than a patient with wild type, CC.

This summary is not intended to identify key or essential features of the claimed subject matter, nor is it intended to be used as an aid in limiting the scope of the claimed subject matter. Furthermore, it is envisioned that alternative embodiments may combine features of two or more of the above-summarized embodiments. Further embodiments, forms, features, and aspects of the present application shall become apparent from the description and figures provided herewith.

### BRIEF DESCRIPTION OF THE DRAWINGS

The concepts described herein are illustrative by way of example and not by way of limitation in the accompanying figures. For simplicity and clarity of illustration, elements illustrated in the figures are not necessarily drawn to scale. Where considered appropriate, references labels have been repeated among the figures to indicate corresponding or analogous elements.

FIG. 1 is a graph illustrating a concentration-time profile of methadone in 38 adolescents over multiple, every 12 hours inter-dose intervals according to an embodiment.

FIG. 2 is a boxplot illustrating opioid use, in terms of morphine equivalent dose per kg, by the children in each inpatient postoperative day (POD) according to an embodiment, and the morphine equivalent dose required was relatively less on POD 1 and 2 than reported in literature and the trend further decreased over the postoperative period with repeated small doses of methadone doses.

FIG. 3 is a boxplot illustrating increasing AAG concentrations in the immediate postoperative period in children undergoing spinal surgery according to an embodiment.

FIG. 4 is a scatter plot illustrating the relationship between acute postoperative pain scores and AAG according to an embodiment.

FIG. 5. is a boxplot illustrating R- and S-methadone drug/metabolite AUC ratio for CYP2B6 rapid metabolizers, normal metabolizers, intermediate metabolizers, and poor metabolizers according to an embodiment.

FIG. 6 is a boxplot illustrating the length of stay in days for FAAH rs3766246 Heterozygous/Homozygous variant and a wild-type according to an embodiment.

FIG. 7 is a boxplot illustrating the mean pain scores for FAAH rs3766246 Heterozygous/Homozygous variant and a wild-type according to an embodiment.

FIG. 8 is a bar chart illustrating the count of nausea and vomiting episodes for POR rs1057868 Heterozygous/Homozygous variant and a wild-type according to an embodiment.

FIG. 9 is a boxplot illustrating the average morphine equivalent dose per day for OPRM1 rs1799971 Heterozygous/Homozygous variant and a wild-type according to an embodiment.

FIG. 10 is a flow diagram depicting a structural model for both R- and S-methadone.

FIG. 11 is a data plot depicting a concentration time profile of R-methadone and its metabolites.

FIG. 12 is a data plot depicting a concentration time profile of S-methadone and its metabolites.

FIG. 13 is a boxplot illustrating the individual variability in the apparent volume of distribution (V2) of R-methadone and S-methadone vs rs17650 genotype after adding AAG concentration as a covariate on V2.

FIG. 14 is a data plot depicting a concentration time profile of AAG in the postoperative period.

FIG. 15 is a graph illustrating different patterns in postoperative AAG levels in patients with different AAG genetic variants (ORM1 rs17650).

FIG. 16 is a boxplot illustrating the relationship between CYP2B6 phenotype (activity score) and CL2 of R-methadone.

FIG. 17 is a boxplot illustrating the relationship between CYP2B6 phenotype (activity score) and CL2 of S-methadone.

FIG. 18 is a graph illustrating the rate ratios of PONV for the rs1038376 variant and the rs2279343 variant.

FIG. 19 is a boxplot illustrating the relationship between CYP2B6 phenotype (activity score) and AUC ratio of R-methadone and S-methadone.

### DETAILED DESCRIPTION

Although the concepts of the present disclosure are susceptible to various modifications and alternative forms, specific embodiments have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

The disclosed embodiments may, in some cases, be implemented in hardware, firmware, software, or a combination thereof. The disclosed embodiments may also be implemented as instructions carried by or stored on one or more transitory or non-transitory machine-readable (e.g., computer-readable) storage media, which may be read and executed by one or more processors. A machine-readable storage medium may be embodied as any storage device, mechanism, or other physical structure for storing or transmitting information in a form readable by a machine (e.g., a volatile or non-volatile memory, a media disc, or other media device).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures unless indicated to the contrary. Additionally, the inclusion of a structural or method feature in a particular figure is not meant to imply that such feature is required in all embodiments and, in some embodiments, may not be included or may be combined with other features.

Intraoperative methadone, a long-acting opioid, is increasingly used for postoperative analgesia, although the optimal methadone dosing strategy in children is still unknown. Use of a single large dose of intraoperative methadone is controversial due to inconsistent reductions in total opioid use in children and adverse effects. Methadone is predominantly bound (80-90%) to AAG, which is an acute phase protein with a half-life of 2-3 days. AAG increases by 2- to 7-fold in response to stressors such as surgery. Such a postoperative increase in AAG levels can potentially decrease free methadone available for receptor binding and clinical analgesia. This role of AAG in the context of perioperative methadone is unexplored.

Multiple small doses of methadone might overcome the shortcomings of a single large intraoperative dose. For instance, multiple dose perioperative methadone regimen in 122 adolescents undergoing PSF at one institution, compared to a historical regimen in that institution without methadone, has been shown to provide the best-in-class outcomes in terms of significantly better analgesia, reduced opioid use, less opioid adverse effects, and shortest hospital stay. Indeed, small repeated doses of methadone intraoperatively and postoperatively provided sustained analgesia and reduced opioid use without respiratory depression. Though this multidose regimen appears to be an effective and safe regimen, in the setting of multimodal analgesia, the pharmacokinetics and the concentration-response relationship in children has never been studied.

The embodiments described below aim to characterize pharmacokinetics, safety, and efficacy of multiple small doses of perioperative methadone in children—integrating the influence of AAG. While not wishing to be bound by any particular theory, it is believed that the multiple small perioperative dose methadone-based multimodal analgesia will result in safe blood methadone concentration (<100 ng/ml) (primary pharmacokinetic endpoint), as well as reduce postoperative opioid use within 72 hours (primary clinical endpoint) and the risk of respiratory depression (secondary clinical end-point) when compared to administration of single-dose methadone in children undergoing PSF. This knowledge on pharmacokinetics, and inter- and intra-individual variations of perioperative methadone will enable optimal use of methadone to improve surgical pain management.

As used herein, the term “AAG” refers to alpha-1 acid glycoprotein. As used herein, the term “ANOVA” refers to analysis of variance. As used herein, the term “Cmax” refers to peak concentration. As used herein, the term “Cmin” refers to trough concentration. As used herein, the term “CPSP” refers to chronic postsurgical pain. As used herein, the term “ECG” refers to electrocardiogram. As used herein, the term “EDDP” refers to 2-Ethylidene-1,5-dimethyl-3,3-diphenylpyrrolidine. As used herein, the term “FDI” refers to Functional Disability Inventory. As used herein, the term “HPLC-MS/MS” refers to high-performance liquid chromatography with tandem mass spectrometry. As used herein, the term “IQR” refers to interquartile range. As used herein, the term “IV” refers to intravenous. As used herein, the term “MEU” refers to morphine equivalent units. As used herein, the term “NMDA” refers to N-Methyl-d-aspartic acid. As used herein, the term “PCA” refers to patient-controlled analgesia. As used herein, the term “PE” refers to pectus excavatum. As used herein, the term “PONY” refers to postoperative nausea and vomiting. As used herein, the term “PSF” refers to posterior spinal fusion. As used herein, the term “QTc” refers to corrected QT interval. As used herein, the term “REDCap” refers to Research Electronic Data Capture database. As used herein, the term “UVD” refers to ultraviolet detection.

According to an embodiment, increasing postoperative AAG levels may correlate with analgesia with methadone. In some embodiments, AAG concentration may contribute to inter-individual variations in methadone's pharmacokinetics and clinical efficacy and safety. According to another embodiment, multiple small doses of methadone may provide better analgesia without respiratory depression. In an embodiment, multiple small doses of perioperative methadone may lower the incidence of chronic persistent surgical pain >3 months after surgery (<5% compared to 36% literature incidence) and improved postoperative functional disability index (FDI) scores.

A correlation between certain genetic characteristics of a person and the effect that administration of methadone has on him or her is demonstrated herein. Knowledge of such correlations can be employed to provide personalized analgesic to persons in need of surgery, thereby increasing the probability of good post-surgical outcomes. One manner of employing such correlations to this end is by performing tests to determine the presence or absence of one or more genetic marker having such a correlation, and selecting a patient care plan based on the presence or absence of such one or more marker. According to an embodiment, making a positive or negative determination whether to administer methadone perioperatively to a patient can be made based on a prediction of the patient's response to perioperative methadone administration. Such a prediction may be made based on a data set that is obtained through preoperative genotype testing. For example, preoperative genotype testing can be administered to a patient in need of surgery as discussed herein to determine the presence or absence of such genetic markers.

### EXAMPLES

Examples related to the present disclosure are described below. In some embodiments, alternative techniques can be used. The examples are intended to be illustrative and are not limiting or restrictive of the scope of the invention as set forth in the claims.

Study Approvals

The studies described herein were conducted at the Riley Hospital for Children after obtaining approval from the Indiana University institutional review board (IRB #1707525204). Written or electronic informed consent was obtained from a parent or a legal guardian of each child and written assent from the child (>14 years of age). This study adheres to the applicable CONSORT guidelines. This study was registered at clinicaltrials.gov (NCT03495388; Principal Investigator: Sadhasivam; Date of registration: 03282018) prior to the start of the trial and any patient enrollment.

### Example 1

### Methods

Study Participants

All children aged 8 to 17.9 years old undergoing PE and PSF surgeries were eligible for this study. Exclusion criteria include the following: allergy to methadone, developmental delay, neurological disorder, liver and/or renal disease, and preoperative pain requiring analgesics.

Analgesia Protocol

As illustrated in TABLE 1, methadone was administered intraoperatively before the incision intravenously and postoperatively through the oral route every 12 hours for 3 to 5 doses at a dose of about 0.1 mg/kg/dose. TABLE 1 provides detailed and standardized multiple perioperative methadone based multimodal analgesia.

TABLE 1
Multidose Methadone-based Multimodal Analgesia Protocol for Posterior
Spine Fusion and Pectus Excavatum Repair in Adolescents

| | Scheduled | PRN |
|---|---|---|
| PreOp | PO acetaminophen (15 mg · kg) 1 dose | |
| | PO celecoxib 1 dose (<45 kg: 100 mg; ≥45 | |
| | kg: 200 mg) | |
| | PO Naloxegol 1 dose (50 kg: 12.5 mg; ≥50 | |
| | mg: 25 mg) | |
| IntraOp | IV Methadone (0.1 mg/kg, max 5 mg) 1 | |
| | dose | |
| | IV Ketorolac (0.5 mg/kg up to 30 mg) 1 | |
| | dose | |
| POD0 | IV Methocarbamol (10 mg/kg) Q8H X 6 | IV morphine (0.05 mg/kg) or |
| | doses | hydromorphone (0.01 mg/kg Q3H |
| | IV ketorolac (0.5 mg/kg) Q6H X 4 doses | PRN |
| | IV Ondansetron (0.1 mg/kg) Q8H x 5 doses | PO Diazepam (2-3 mg) Q6H PRN |
| | PO methadone Q12H X 1 dose - evening | IV Nalbuphine Q6H PRN |
| | PO Acetaminophen Q6H | Scopolamine patch Q72H PRN |
| POD1 | PO Oxycodone (0.1 mg/kg, max 5 mg) | IV morphine/hydromorphone Q3H |
| | Q6H | PRN |
| | PO Naloxegol Q24H | PO Diazepam Q6H PRN |
| | PO methadone Q12H X 2 doses | IV Nalbuphine Q6H PRN |
| POD2 | PO ibuprofen (10 mg/kg) Q6H | PO Ondansetron Q6H PRN |
| | PO methadone Q12H X 1 dose | PO Diazepam Q6H PRN |
| | PO methocarbamol Q8H | |
| | PO Oxycodone (0.1 mg/kg) Q6H | |
| | PO Naloxegol Q24H | |

PRN: Pro Re Nata (as needed)
PO: Per Oral
IV: Intravenous
SC: subcutaneous
PreOp: Preoperatively
IntraOp: Intraoperatively
POD: Post-operative day
Pectus excavatum patients receive a thoracic epidural analgesia intraoperatively and postoperatively with an infusion of 0.2% ropivacaine, that is continued until POD2, in addition to the above multimodal analgesic protocol.

Specimen Collection, Storage, and Analysis

Blood samples were collected at specific time windows, including at 10-30 minutes, 2-6 hours, 10-12 hours after dose 1 and 3, 1-2 hours, and 10-12 hours after dose 2 through a designated intravenous line in situ based on the pharmacokinetics of the drug concentration-time profile to catch both the peak and the trough concentrations. Some samples collected for oxycodone pharmacokinetic analysis were also used for methadone assays. A validated high-performance liquid chromatography with tandem mass spectrometry (HPLC-MS/MS) chiral assay was used to measure the concentration of R- and S-methadone and metabolites R- and S-EDDP (2-ethylidene-1,5-dimethyl-3,3-diphenylpyrrolidene) while a HPLC-ultraviolet detection (UVD) assay was used to determine the AAG. The total methadone concentrations were considered as the sum of measured R- and S-methadone concentrations.

Pharmacokinetic Measures

The peak concentration (Cmax) and trough concentration (Cmin) of the individual enantiomers and the total drug were obtained from the concentration time profiles of individual patients. Peak metabolite to parent drug ratios of R-, S-, and total methadone were calculated. Concentrations of AAG were measured at all time points of methadone and metabolites measurement.

Pharmacodynamic Measures

To standardize the comparison across the observed population, all opioids were converted to a morphine equivalent dose using the following morphine equivalency: intravenous (IV) hydromorphone=5, IV methadone=3, oral methadone=3, IV morphine=1, oral oxycodone=2, IV fentanyl=100, oral hydrocodone=0.33, and IV nalbuphine=1. The clinical outcomes were pain scores and adverse events, such as PONV, respiratory depression, and heart-rate corrected QT (QTc) interval prolongation. Pain scores were collected every 2 to 4 hours using a self-reported numeric rating scale ranging from 0 to 10. Episodes of PONV and respiratory depression were recorded from clinical monitoring, reports, and records. Respiratory depression was defined as oxygen saturation of less than 90% and/or a respiratory rate of less than 8 per minute for >1 minute while breathing room air. Chronic disability was measured using Functional Disability Inventory (FDI) preoperatively and at 3, 6, and 12 months postoperatively. The FDI is a validated questionnaire with 15 questions administered to children to assess chronic persistent surgical pain (CPSP) with each question answered on a 5-point Likert scale. Electrocardiograms (ECGs) were obtained on all patients once preoperatively and once postoperatively to obtain corrected QT interval (QTc) using Bazett's formula. QTc intervals were measured manually in 20% of the ECGs to verify the accuracy of the machine-reported values. QT interval prolongation was defined as QTc interval greater than 460 ms (all patients <15 years), 450 ms (males >15 years), and 470 ms (females >15 years) with a plan to stop methadone if the QTc interval was prolonged to reduce the risk for the ventricular tachycardia, torsades de pointes.

Statistical Analysis

Besides the primary pharmacokinetic endpoint of methadone levels, clinical efficacy (opioid use, pain scores) and safety (QTc lengthening, PONV and respiratory depression) were analyzed. The clinical efficacy (25% reduction in morphine equivalent dose) and safety (25% reduction in respiratory depression) endpoints with the multiple small methadone regimen were compared to an earlier published historical cohort of adolescents undergoing PSF. The following secondary analyses were also conducted: 1. association between the pharmacodynamic and the pharmacokinetic parameters (peak and trough concentrations of R-, S-, and total methadone, R-, S-, and total EDDP, AAG concentrations), methadone dose, morphine equivalent dose, and demographic factors; 2. association of pharmacodynamic parameters (pain scores, the difference in QTc, episodes of respiratory depression and PONV) with the closest enantiomeric and total methadone and metabolite as well as AAG concentrations. Closest concentrations were assumed as concentrations of methadone, metabolite, or AAG available from specimens collected at time points within 1 hour (before or after) of the time of collection of the pharmacodynamic parameters. Pain scores, postoperative QTc, and the difference in QTc were considered as continuous variables, while respiratory depression and PONV were considered as binary variables; and 3. association between pharmacodynamic parameters (average pain scores, FDI score, postoperative QTc, respiratory depression, and PONV) with summary measures—average morphine equivalent dose, methadone dose, AAG concentration, peak and trough concentrations, and metabolite ratio.

Descriptive analysis was done for all the measured parameters along with the primary interest, total morphine equivalent dose at 72 hours. For the rest of the analysis, linear regression was done for the continuous dependent outcomes and logistic regression was done for the binary dependent outcomes. In the event of the presence of multiple independent variables with significant relationship to the dependent outcome on simple regression analyses, a multiple regression analysis was used to analyze the effect of confounders. Adjusted R² values were used whenever more than 1 independent variable was tested for association. Residual versus fitted plots and Q-Q plots were checked for all linear regression models. ANOVA with post-hoc Tukey's test was used to determine the change in average AAG concentrations and morphine equivalent dose among days 1, 2, and 3 in the post-operative period. A linear mixed effect model was used to analyze the pain outcome. The model included demographic variables, time, surgery type, methadone concentration and AAG as fixed effect; and individual random effect (i.e. random intercept). P values were calculated using the Kenwar-Roger method under R package ImerTest. Paired samples t-test was performed to check for increase in QTc in the perioperative period.

Since there were no randomized control group in this Example due to lack of equipoise in the institution in which the embodiment was performed, the incidence of respiratory depression and the amount of morphine equivalent dose required were compared with published literature with similar study population with the sample size was based on 25% reduction in morphine equivalent dose and respiratory depression. The historical cohort had a mean morphine equivalent dose of 277 mg (0-72 hour with 0.1 mg/kg intraoperative methadone), and multi-dose methadone regimen in this Example was associated with a mean morphine equivalent of 113.25 with a standard deviation of 28.4. Using the one-sample t-test post-hoc, a sample size of 38 will yield a power of over 95% and controls the type I error at 5%. All analyses were conducted using the statistical software R.

### Results

Patients

A total of 38 children (13 had PE and 25 had PSF) were recruited as shown in TABLE 2.

TABLE 2
Baseline Characteristics, Pharmacokinetics, and Pharmacodynamics

| | Values (median or # of patients) | | | |
|---|---|---|---|---|
| | Type of surgery | | | |
| | Pectus excavatum | | Idiopathic scoliosis | |
| | repair (n = 13) | | fusion (n = 25) | |
| Age (years) | 15.6 | (13.6-16.3) | 14.2 | (12.8-16) |
| Weight (kg) | 57.3 | (50.2-67.9) | 52.7 | (47.9-58.3) |
| Height (cm) | 172 | (168-179) | 161 | (157-166) |
| BMI | 18.1 | (17.2-20.6) | 20.3 | (17.9-22.6) |
| Race | | | | |
| Caucasian | 11 | | 20 | |
| Hispanic | 1 | | 0 | |
| African American | 0 | | 5 | |
| Unknown | 1 | | 0 | |
| Biological sex (Female:Male) | 2:11 | | 18:7 | |
| Methadone dose (mg/kg/day) | 0.18 (0.17-0.19) | | | |
| Oxycodone (mg/kg/day) | 0.23 (0.19-0.26) | | | |
| Hydromorphone (mg/kg/day) | 0.014 (0.011-0.020) | | | |
| Morphine equivalent dose 0-72 hour (mg) | 120.2 (98.5-131.8)) | | | |
| Average morphine equivalent dose (mg/kg/day) | 0.66 (0.59-0.74) | | | |
| Cmax (ng/mL) | | | | |
| R-methadone | 11.0 (8.2-16.1) | | | |
| S-methadone | 13.1 (10.3-24.2) | | | |
| Total methadone | 24.7 (19.2-40.8) | | | |
| Cmin (ng/mL) | | | | |
| R-methadone | 1.50 (1.2-2.2) | | | |
| S-methadone | 2.38 (1.52-3.82) | | | |
| Total methadone | 4.09 (2.74-6.4) | | | |
| AAG (ug/mL) | 95.2 (75.6-129.1) | | | |
| QTc (ms) | | | | |

| | |
|---|---|
| Preoperative | 416 (397-427) |
| Postoperative | 431 (414-440) |
| Average pain score | 3.53 (2.73-4.80) |

Pharmacokinetic and Pharmacodynamic Parameters

Methadone was administered at a median dose of 0.18 (interquartile range [IQR] 0.17-0.19) mg/kg/day as shown in TABLE 2. Though per kg methadone dose was similar for the two surgical types, median total opioid usage was higher following PE compared with PSF (2.6 versus 2.1 mg/kg; P=0.028). The median morphine equivalent dose 0-72 hour (mg) was 120.2 (98.5-131.8). The median opioid usage was minimal, 0.66 (IQR 0.59-0.75) mg/kg morphine equivalents/day. Opioid usage decreased from postoperative day 2. The median usage of the common rescue opioids was not different between the surgeries (0.03 mg/kg morphine equivalents).

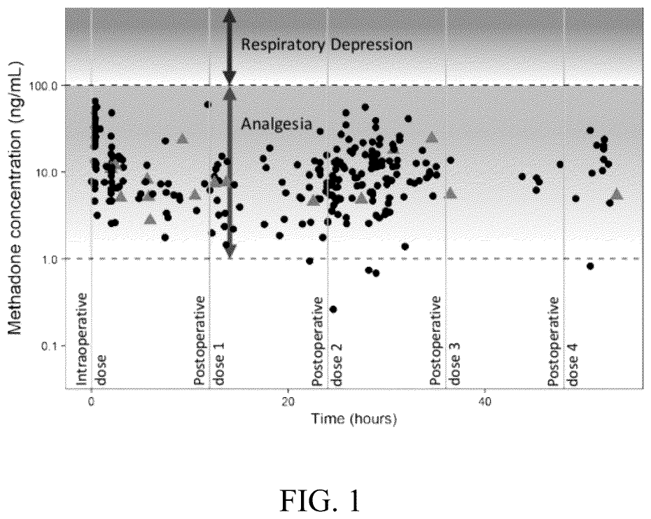

As illustrated in TABLE 2, the average cumulative dose of methadone, oxycodone and hydromorphone over the entire perioperative period were 0.37, 0.52 and 0.025 mg/kg body weight respectively. Methadone contributed to about 47.8% of the total morphine equivalent used and did not differ between the surgeries (P=0.275). The median contribution of oxycodone to total morphine equivalent were 54.4% and 46.1% (P=0.013) for PE and PSF; the median hospital stay was 4 and 3 days respectively (P=0.002). Referring now to FIG. 1, the concentration-time profiles of all the individuals for total methadone is shown as a dot plot. The lines represent methadone dosing time points. The dots represent concentration of total methadone (R+S enantiomers) measured. Smaller methadone doses repeated every 12 hours maintained analgesic concentrations beyond 24 hours. None of the children attained concentrations that could cause respiratory depression (>100 ng/ml). The triangles represent concentrations measured close to an episode of postoperative nausea or vomiting. The median peak (Cmax) was 24.7 (IQR 19.2-40.8) ng/mL and the median trough (Cmin) was 4.09 (IQR 2.74-6.4) ng/mL.

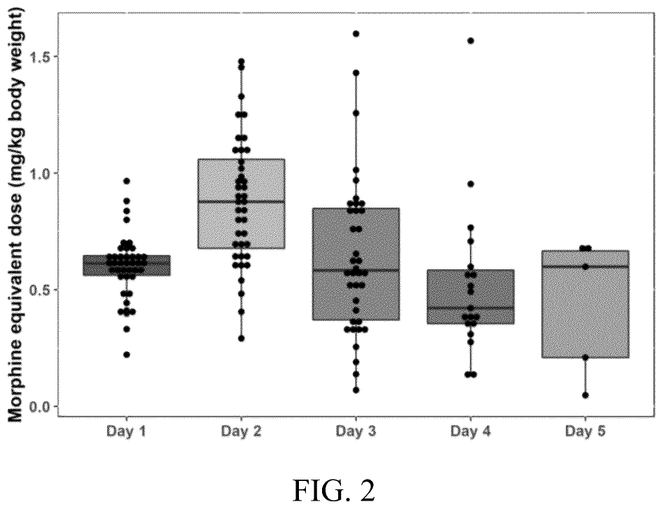

Referring now to FIG. 2, including methadone doses, median (interquartile range) morphine equivalent units (MEU)/kg for postoperative days 1-5 were 0.61 (0.56-0.65), 0.88 (0.68-1.06), 0.58 (0.37-0.85), 0.42 (0.35-0.58), and 0.59 (0.21-0.67), respectively. The morphine equivalent dose required was relatively less on POD 1 and 2 than reported in literature and the trend further decreased over the postoperative period with repeated small doses of methadone doses. Opioid usage steadily decreased from the second postoperative day as illustrated in FIG. 2.

Functional Disability Inventory

The median (IQR) FDI score in the preoperative period was relatively low, 6.5 (0-13). The median FDI scores further decreased and improved gradually to 4 (0-9.5), 2 (0-4) and 0 (0-3.5) at the 3, 6, and 12 months postoperatively. On a univariate analysis, the 3-month FDI score was directly related to the preoperative FDI score, morphine equivalent dose, and average AAG (P<0.05). On a multiple linear regression analysis, only average AAG remained significant for 3 month FDI scores (P=0.017; R2=0.22).

AAG

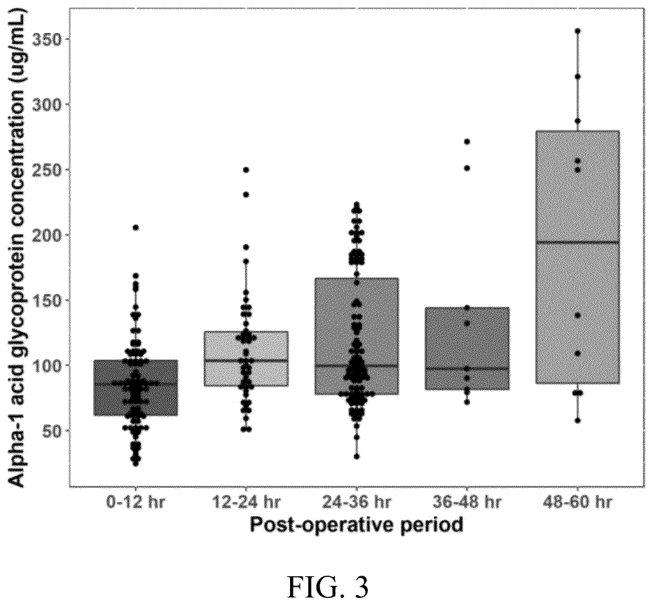

Referring now to FIG. 3, the concentration of AAG increased steadily over the postoperative period in most children and almost doubled at 48 hours after surgery (median 193.9 IQR 86.3 to 279.5 μg/mL) from intraoperative levels (median 87.4 IQR 70.6 to 115.8 μg/mL, P<0.001), and change of AAG from intraoperative period to 48 hours post operatively correlated with R-EDDP (P<0.001), S-EDDP (P<0.001) levels, and pain scores (P=0.008). An ANOVA test followed by a post-hoc Tukey's test showed significant postoperative increases in AAG (P<0.001). The average AAG of PE and PSF surgeries (104.7 and 113.7 μg/mL, respectively) did not differ significantly (P=0.090). Univariate regression analysis showed that AAG was associated with both the R-EDDP and S-EDDP concentration (P<0.001; R2=0.074 for R-EDDP and 0.091 for S-EDDP) but was not associated with methadone concentration (P >0.149 for R-methadone and P=0.098 for S-methadone).

Pain

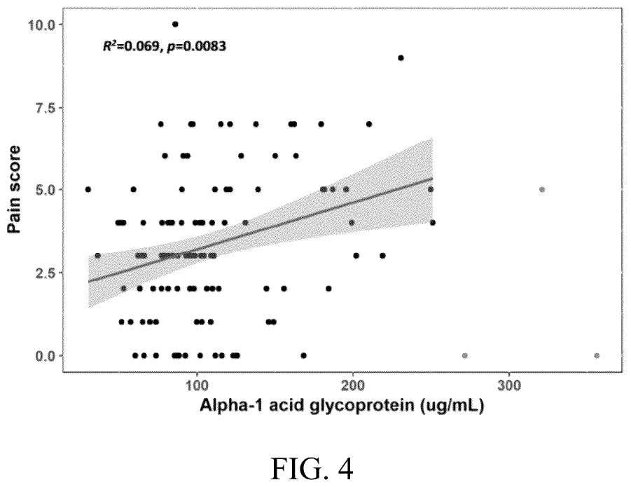

Pain scores (median, IQR) for postoperative days 1-5 were 3.5 (2.3-5.1), 3.4 (2.9-4.6), 3.7 (2.0-5.2), 3.8 (2.7-5.0), and 3.7 (3.3-4.4), respectively. Average pain scores during the entire in-hospital stay were lower for the PE (3.60) compared with PSF (3.95; P=0.036). In univariate analyses, there were no associations found between the R-, S-, and total methadone in relation to the individual pain scores. However, R-, S-, and total EDDP concentrations were inversely associated with the pain scores (P=0.028, 0.038 and 0.033 respectively, R2=0.04 in all cases). Initially, AAG was not associated with pain scores (P=0.218, R2=0.015). However, after excluding one patient who had a few outlying concentrations of AAG (>300 μg/mL), the correlation between AAG and pain scores was significant (P=0.008, R2=0.069) as shown in FIG. 4. In other words, AAG concentrations directly correlated with the postoperative scores. Average daily pain scores were positively correlated with average daily AAG concentrations (P=0.037, R2=0.065). No other relationships were found between pain scores and methadone dose, Cmax or Cmin, metabolite ratio, opioid use, or the demographic factors as shown in TABLE 3. Performing longitudinal analysis using a linear mixed model, AAG was again observed to be associated with increased pain scores (P=0.042). With other variables unchanged, 1 unit increase of AAG was associated with 0.01 unit increase of pain score.

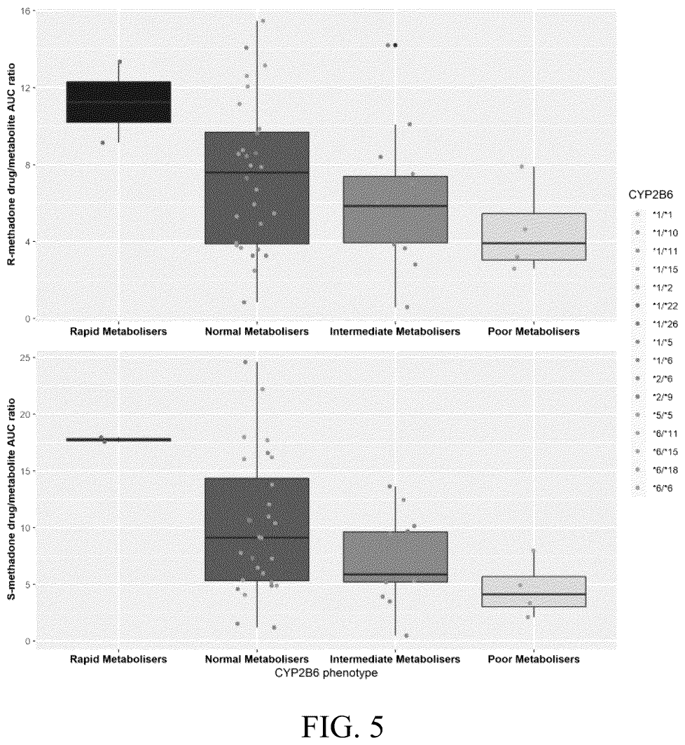

Referring now to FIG. 5, preliminary CYP2B6 Data from 50 PSF adolescents: Methadone dosing algorithms in children could improve its analgesia and safety, especially with repeated doses. Adolescents that were poor metabolizers (*6/*6) had >2-fold lower R- and S-methadone drug/metabolite AUC ratios, compared with normal (*1/*1) or rapid metabolizers (*1/*22). Intronic SNP, rs8192719 (21563 C>T) is one the three CYP2B6 SNPs that were associated with methadone fatalities in adults; this SNP was associated significantly with methadone/metabolite AUC ratio in 50 PSF adolescents. Besides PK variability, for the first time, novel PD associations are identified between methadone-induced PONV and CYP2B6 SNPs, rs1038376 (P=0.005), rs10853744 (P=0.024), rs7250601 and rs7250991 (P=0.022), as well as maximum post-surgical pain (r511882424, P=0.007) to enable precision methadone dosing along with AAG levels.

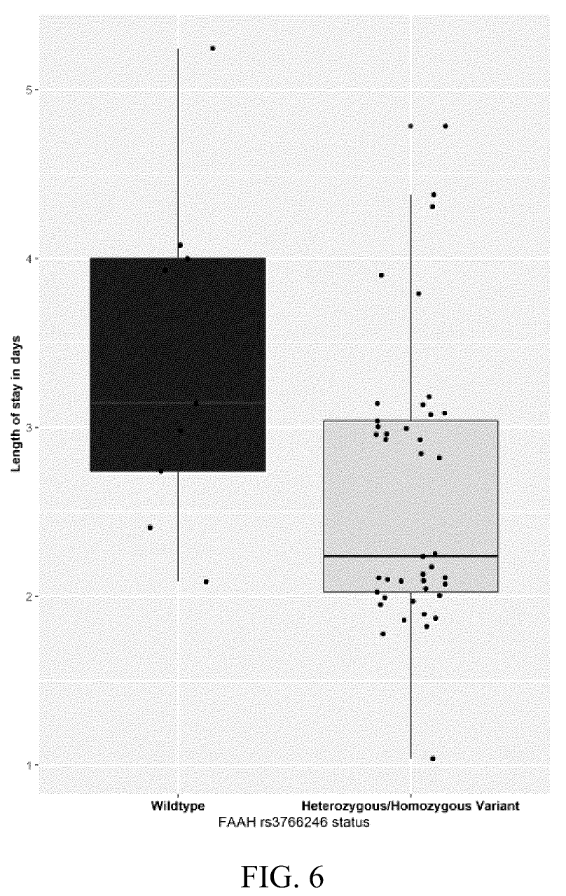

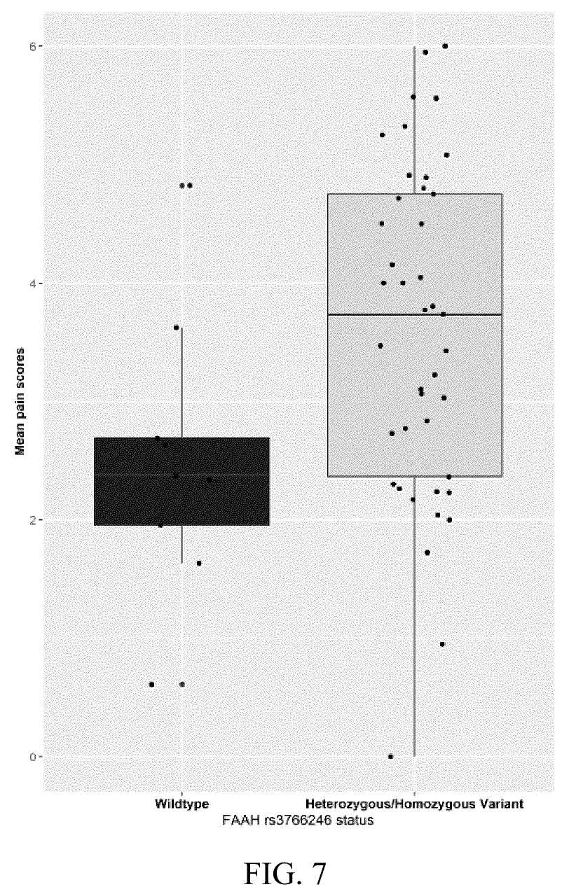

Referring now to FIG. 6 and FIG. 7, in adolescents undergoing PSF that received methadone, compared with the wild type, the homologous/heterologous variants of SNPs of FAAH rs3766246 (P=0.006) and rs4141964 (P=0.005) had 15-20% lower in-hospital opioid use. Adolescents with the wild type of rs3766246 had more PONV (P=0.008) and 1 day longer hospital stay than others (2.6±0.8 days; P=0.04).

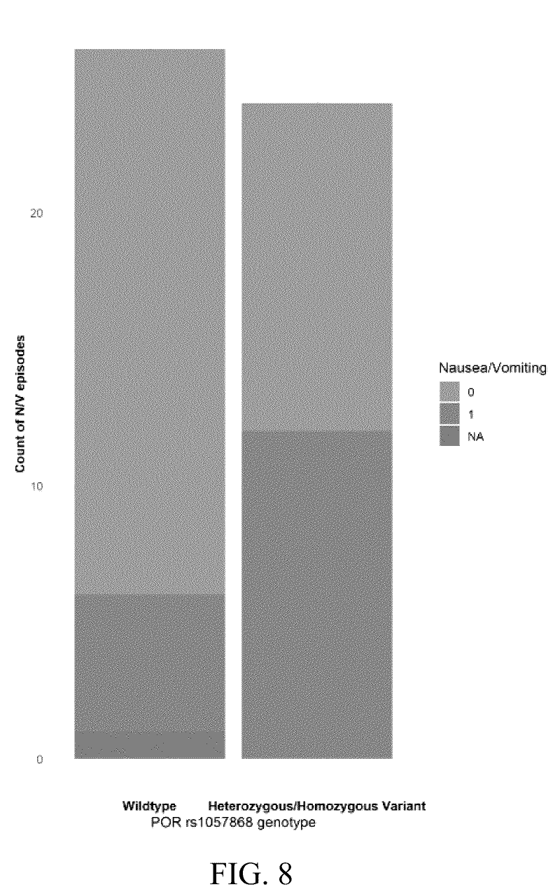

Referring now to FIG. 8, in the 50 PSF adolescent cohort, POR rs1057868 TT and CT genotypes (with anticipated higher CYP3A4 activity, higher methadone inactivation and higher requirements of non-methadone opioids) was associated with higher incidence of PONV than the wild type, CC (P=0.026); the higher total opioid use in those with PONV (P=0.005, R2=0.21) supports this association.

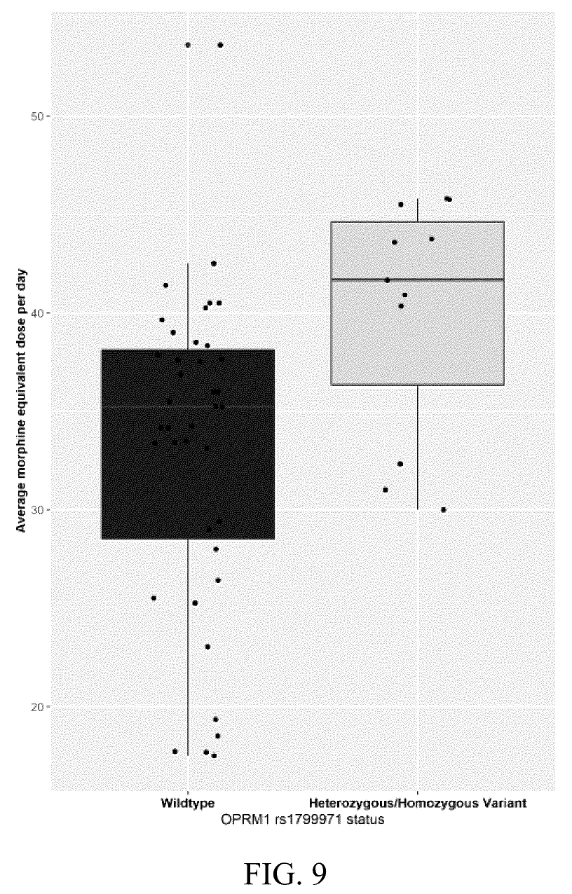

Referring now to FIG. 9, in 50 adolescents undergoing PSF that received methadone, those with OPRM1 rs1799971 GG and GA genotype (40.1±6.1 mg) had 20% higher average in-hospital opioid use than AA genotype (33.2±8.0, P=0.0056). Adolescents with AA have more PONV (P=0.011), and longer length of stay (LOS) (P=0.036). OPRM1 rs1799971 is also associated with susceptibility to dependence. The largest (n>100,000) and recent genome-wide association study in adults identified rs1799971 as associated with opioid use disorder (OUD), with independent replications (genome-wide significance; P=1.51×10⁻⁸).

Additionally, ABCB1 in a multi-gene model predicts methadone's dose-response in MMT. In the 50 adolescents undergoing PSF that received methadone, higher risk of PONV was associated with ABCB1 rs1128503 (P=0.008) and rs1045642 (P=0.037).

Further, in 50 adolescents undergoing PSF, DRD2 rs1800497 (TaqA1 ofANKK1) was associated with PONV (P=0.001) and longer hospital stay (P=0.002). DRD2 rs1800497 has been associated opioid dependence and opioid use disorder in addition to methadone dose variations.

Respiratory Depression

No child in this Example had respiratory depression (95% Wilson binomial confidence [0, 0.09]). In addition, as shown in FIG. 1, no child in this Example achieved methadone concentrations (>100 ng/ml) previously found to be associated with respiratory depression (95% Wilson binomial confidence [0, 0.09]).

PONV

There were 18 episodes of PONV seen in 12 patients as illustrated in FIG. 1. Binomial regression showed a relationship between morphine equivalent dose and PONV (P=0.021, OR=2.9; 95% CI=1.3-8.2). Since, some patients had multiple PONV episodes over different postoperative days, a univariate linear regression showed a relationship between morphine equivalent dose and episodes of PONV (P=0.005, R2=0.21). The type of surgery did not remain significant in multivariable analysis as illustrated in TABLE 3.

TABLE 3
Correlations of Clinical Outcomes With Pharmacokinetic And Demographic Measures

| Pharmacokinetic | Pain scores | | Postoperative QTc | | Nausea/Vomiting | | |
|---|---|---|---|---|---|---|---|
| measure | R² | P value | R² | P value | OR (CI) | | P value |
| R-methadone | 0.002 | 0.631 | 0.012 | 0.588 | | | |
| S-methadone | 0.007 | 0.356 | 0.029 | 0.392 | | | |
| Total methadone | 0.005 | 0.445 | 0.004 | 0.740 | | | |
| R-EDDP | 0.042 | 0.028 | 0.020 | 0.477 | | | |
| S-EDDP | 0.038 | 0.038 | 0.029 | 0.393 | | | |
| Total EDDP | 0.040 | 0.033 | 0.026 | 0.419 | | | |
| AAG | 0.069 | 0.008 | 0.175 | 0.042 | | | |
| Morphine equivalent | 0.013 | 0.507 | <0.001 | 0.876 | 2.92 | (1.27-8.15) | .02 |
| dose per kg per day | | | | | | | |
| Methadone dose | 0.002 | 0.776 | 0.006 | 0.657 | 3.1e3 | (3.2e−20-8.4e+29) | 0.77 |
| per kg per day | | | | | | | |
| Average AAG | 0.065 | 0.037 | 0.062 | 0.598 | 1.01 | (0.99-1.03) | 0.82 |
| Age | 0.016 | 0.459 | 0.028 | 0.331 | 1.26 | (0.85-1.97) | 0.27 |
| BMI | 0.012 | 0.511 | 0.003 | 0.735 | 0.97 | (0.81-1.14) | 0.76 |
| Biological Sex | 0.0001 | 0.946 | 0.126 | 0.034 | 0.52 | (0.12-2.07) | 0.36 |
| Ethnicity | 0.101 | 0.324 | 0.129 | 0.210 | —* | | >0.5* |
| Type of surgery | 0.022 | 0.384 | 0.143 | 0.486 | 0.21 | (0.04-0.89) | .04 |

QTc Interval Prolongation

The median (IQR) of preoperative and postoperative QTc intervals were 416 (397-427) ms and 427 (413-439) ms respectively. Though the postoperative QTc was longer (Median 9 IQR-10, 28 ms) than the preoperative QTc interval, (P=0.016), it was not clinically significant. In other words, no significant QT prolongation occurred. The median QTc interval in boys was longer than that for girls (P=0.022 preoperative; P=0.015 postoperative). The postoperative QTc was associated with the closest AAG concentrations in a univariate regression analysis (P=0.042, R2=0.18), as displayed in TABLE 3.

Comparison with Historical Cohort

The mean morphine equivalent dose in the first 72 postoperative hours in this Example was 120.2 mg (95% CI: 98.5-131.8) and was about 50% less than in a study with a similar cohort of adolescents undergoing PSF9 receiving PCA opioids without intraoperative methadone [274 mg (95% CI: 192-356); 58.67% opioid use reduction, P-value <0.001], a single intraoperative methadone of 0.1 mg/kg [277 mg (95% CI: 185-369); 59.12% opioid use reduction, P-value <0.001 (compared to the embodiment of this Example)=<0.001], 0.2 mg/kg (95% CI: 139-291); 47.81% opioid use reduction, P-value <0.001] and 0.3 mg/kg [221 (95% CI: 141-301); 48.76% opioid use reduction P-value <0.001]. Moreover, that study, even with a small sample size (n=31 receiving intraoperative methadone) reported higher incidences of postoperative respiratory depression (7 out of 31, 22.6% in the first 48 hours), possibly due to higher postoperative opioid use. In this Example of 38 children receiving multiple dose of methadone, 0% of respiratory depression was observed. This difference and reduction in one of the serious opioid adverse effects, postoperative respiratory depression is clinically and statistically significant (Fisher exact test, P-value=0.01155; OR=Infinity (95% CI: 1.598-Infinity).

### Discussion

This first pharmacokinetic study of novel multiple perioperative methadone in children undergoing PSF and PE demonstrated safe and effective blood methadone levels with opioid sparing sustained analgesia without respiratory depression. Better clinical efficacy and safety of this methadone-based regimen has been observed in terms of significant opioid sparing, pain control, and shorter hospital stay compared to a non-methadone based analgesic regimen. In the current Example, the median morphine equivalent dose (mg) in the first 72 postoperative hours was nearly half the amount used in a study with a single intraoperative methadone use in a similar population, illustrating the efficacy of this multidose methadone regimen. The median morphine equivalent dose 0-72 hour (mg) was less with this multidose methadone regimen compared to another published analgesic regimen. In addition, the methadone dose used in this Example is approximately half of what is typically used in other studies. Another adolescent PSF study with a single larger intraoperative methadone dose reported higher postoperative opioid use (morphine equivalents 0.54, 1.08, and 1.29 mg/kg compared with 0.6, 0.88, and 0.64 mg/kg, respectively) on postoperative days 1, 2, and 3 reported in the current Example. These highlight non-opioid sparing effects of single intraoperative methadone compared to multiple perioperative doses of methadone in children. None of the 38 children in this Example had blood methadone concentration above 100 ng/ml and experienced postoperative respiratory depression, highlighting the relative safety of this regimen. In adolescents that received intraoperative methadone alone, a high incidence of respiratory depression (22.6% in the first 48 hours), was reported which is possibly due to about 50% higher opioid use than observed in the current Example.

Methadone's pharmacokinetics (PK) is described using a three compartment model with a long elimination half-life of 44 hours, but a short redistribution half-life. After a single bolus dose, rapid redistribution and the consequent fall in blood levels can result in rapid termination of effect, especially with small doses. Even postoperative infusion after intraoperative methadone was suggested to overcome this shortcoming. At large doses, termination of effect reflects the elimination half-life, but this could be associated with higher adverse events. This Example demonstrates that multiple small doses of methadone is an effective and clinically feasible dosing strategy.

Blood methadone concentrations observed in this Example were lower than the reported minimal effective analgesic concentrations in a small adult study conducted more than three decades ago prior to an era of multimodal postoperative analgesia. In this Example, multidose methadone was used as part of a multimodal regimen including intraoperative ketamine and remifentanil infusions for PSF and thoracic epidural analgesia for PE patients. With this multimodal regimen with multiple small doses of methadone, effective and sustained postoperative analgesia was feasible at much lower blood methadone levels than previously reported minimum effective analgesic levels. Lack of sustained analgesia with a single dose of intraoperative methadone in children could be related to the relatively shorter half-life of methadone in children compared with adults.

AAG concentrations steadily increased from intraoperative levels and almost doubled at 48 hours after surgery. Since AAG binds to methadone, its concentrations determine the amount of free methadone. Interestingly, AAG concentrations were inversely related to pain relief, possibly from an inverse relationship between AAG and free methadone. AAG is an acute-phase protein and its concentration increases in the immediate post-operative period. The AAG-bound-to-unbound ratio of methadone has been shown to directly relate to the AAG concentration in a linear or an exponential relationship. As used herein, the term “linear relationship” refers to an association between two variables that when subjected to regression analysis and plotted on a graph forms a straight line. In linear relationships, the direction and rate of change in one variable are approximately constant with respect to changes in the other variable. As used herein, the term “exponential relationship” refers to a relationship in which a constant change in the independent variable gives the same proportional change (that is, percentage increase or decrease) in the dependent variable. Without intending to be bound by any particular theory, it is believed that free methadone is in equilibrium with the bound methadone and may be responsible for the efficacy and adverse effects. AAG concentrations and trends varied considerably among individuals postoperatively consistent with published results. Increasing postoperative AAG emphasize the importance of AAG in modulating perioperative methadone effects.

Methadone is not utilized preoperatively frequently because of its long elimination half-life, which could delay recovery in the event of respiratory depression. A large observational study with an intraoperative methadone of 0.14 mg/kg and postoperative opioids reported a 37% incidence of respiratory depression; other studies reported the incidence ranging from 0 to >10% with intraoperative methadone in adult and pediatric surgical populations. However, as demonstrated in the present Example, despite the use of repeated doses of methadone, no patient had respiratory depression, and blood methadone levels remained below 100 ng/mL. Besides providing safe and sustained opioid-sparing analgesia, multiple small doses of methadone may be cost-effective in the current setting of value-based care. For instance, a multi-dose methadone regimen has been shown to eliminate the need for patient controlled analgesia (PCA) in adolescents undergoing PSF and helped to achieve the shortest hospital stay following PSF. This led to substantial cost savings ($10,000/patient). Methadone is also >10-fold less expensive compared with opioid PCA.

PONV was observed in 31.6% of patients in this Example—similar to other studies (33% to 38% incidence). In this Example, PONV was positively associated with cumulative opioid use, even at lower analgesic concentrations of methadone. Genetic predisposition and efficacy of anti-emetics may help identify and reduce methadone-related PONV risks.

Methadone, especially its S enantiomer, is known to cause QT prolongation with both the dose and duration of methadone directly influencing the extent of QT lengthening. With chronic methadone treatment, EDDP concentrations were associated with QT prolongation. None of the children in this Example had clinically significant QT prolongation. The postoperative QTc was directly related to the closest AAG concentration—indicating that the free methadone influences the amount of QT lengthening. Given the high interindividual variability, it illustrates the importance of AAG and precision methadone dosing.

Functional disability, an indicator of CPSP, improved over the 3, 6, and 12 postoperative months in this Example. The 3-month FDI score correlated with average AAG level. Without intending to be bound by any particular theory, it is believed that this correlation may possibly be due to an increase in physiological stress, trauma, and acute surgical pain. The incidence of CPSP and FDI scores in this Example is less than reported in an earlier study that did not use perioperative methadone and ketamine. Without intending to be bound by any particular theory, it is believed that lower FDI scores could be due to the use of intraoperative NMDA antagonists, methadone, and ketamine, reducing opioid-induced hyperalgesia and acute opioid tolerance. Postoperative methadone use also helped to reduce acute surgical pain and possibly mitigated the transition to chronic pain.

The strengths of the present embodiment are its prospective nature, use of multiple small perioperative doses of methadone, and simultaneous pharmacokinetic analyses including implications of increasing postoperative levels of AAG on methadone's effects. Embodiments disclosed herein demonstrate effective and sustained analgesia with multiple small doses of methadone in children without increasing the risks of respiratory depression frequently reported with larger intraoperative doses of methadone in children and adults.

Novel multiple small perioperative methadone doses resulted in safe and lower blood methadone concentration levels, <100 ng/ml, a threshold previously associated with respiratory depression, and provided effective and sustained analgesia without respiratory depression in children undergoing painful inpatient surgeries. Increasing postoperative AAG influenced methadone's effects, perhaps by lowering free methadone. This Example provides clinical and pharmacokinetic support for safe and effective perioperative multiple doses of methadone use in children, which is superior to previously published strategies, including a single intraoperative methadone dose. This evidence would facilitate wider adoption of perioperative methadone to safely provide sustained analgesia.

The combinatorial pharmacokinetic and polygenic variations in CYP2B6, POR, OPRM1, FAAH, ABCB1 and DRD2 genes along with varying concentrations of AAG levels help predict methadone-induced adverse effects, pain relief, in-hospital opioid use, length of hospital stay, chronic surgical pain besides selection of precise methadone dose to avoid life-threatening complication of methadone, respiratory depression.

### Example 2

### Pharmacokinetic Modeling of R- and S-Methadone and their Metabolites to Study the Effects of Various Covariates in Post-Operative Children

This Example's objective was to identify the relevant sources of variability in the pharmacokinetics of R- and S-methadone including the physiological parameters and the genotype in children undergoing major surgeries.

### Methods

Study Participants

All children and adolescents aged 8 to 17.9 years old undergoing orthopedic surgeries, PE and PSF surgeries were eligible for the current embodiment. Exclusion criteria include the following: allergy to methadone, developmental delay, neurological disorder, liver and/or renal disease, and preoperative pain requiring analgesics.

Dosing Schedule and Sample Collection

The first dose of racemic methadone was administered intravenously intraoperatively and thereafter postoperatively every 12 hours orally for a total of 4 to 6 doses at a median dose of 0.087 mg/kg (IQR 0.069-0.094). Oral doses were either given as a tablet or a suspension depending on the patient's convenience. Blood samples were collected at specific time windows 10-30 minutes, 2-6 hours, 10-12 hours after dose 1 and 3, 1-2 hours, 10-12 hours after dose 2 for the first 40 patients. The time schedule was later modified to collect more samples through the 1^{st} and 3^{rd} inter-dose interval without samples through the 2n d interdose interval. This was done to aid in the optimal spread of the collected concentration time-points and improvement of estimation of absorption parameters of the oral doses. Some of the samples collected in a parallel study were also used whenever available with the consent of the patient to aid with the better estimation of the terminal elimination phase.

Analytical Method

Quantification of Enantiomers of Methadone and EDDP

R-methadone and S-methadone and their metabolites R- and S-EDDP (2-ethylidene-1,5-dimethyl-3,3-diphenylpyrrolidene) were measured by a validated high-performance liquid chromatography with tandem mass spectrometry (HPLC-MS/MS) chiral assay while AAG was measured by a HPLC-ultraviolet detection (UVD) assay. The lower limit of quantification (LLOQ) was 0.15 ng/mL for the drug and the metabolite. Coefficients of variation (“CV”) were <15% for all the QC for all these analytes. The LLOQ for AAG was 20 μg/mL and the CV were <10% for all the QC of AAG. The total methadone concentrations were considered as the sum of measured R- and S-methadone concentrations.

Genotyping

Subjects' whole-genome sequencing BAM and corresponding VCF files were ingested into LifeOmic's (Indianapolis, IN, USA) Precision Health Cloud (PHC) for PGx analysis. Aldy v3.0 was operationalized in the PHC to genotype pharmacogenomic star alleles and diplotypes for CYP2B6, CYP2C8, CYP2C19, CYP2D6, CYP3A4, and CYP3A5 using the BAM files as input for Aldy. In the PHC, JupyterLab notebook was used to extract genotypes of interest not covered by Aldy from the VCF files. Both Aldy diplotype calls and genotypes of interest not covered by Aldy were outputted in .csv file format for further analysis. Genes coding for CYP enzymes were classified as their corresponding phenotypes —/IM/PM (ultra-rapid, normal, intermediate and poor metabolizers) and were used for analysis. Other single nucleotide polymorphisms (“SNPs”) were analyzed using the dominant, additive or recessive models.

PK Modeling

R- and S-methadone were modeled individually using non-linear mixed effects modeling in the software NONMEM 7.4. The first-order conditional estimation method with η-ε interaction (FOCE-I) was used throughout the model building process. The dose of the drug, concentrations of the drug and the metabolite were converted to their molar counterparts to aid in the accurate estimation of the pharmacokinetic parameters. Perl-Speaks-NONMEM v.4.9.0, R v.4.0.4 (R Foundation for Statistical Computing) with the Xpose4 package v.4.6.0, RStudio v1.4 and Pirana v.2.9.9 were used for data exploration, visualization, diagnostics and automation. Measured plasma concentrations that were below the LLOQ were dealt with using the M1 method (discarding concentrations below the LLOQ and estimate the PK parameters using the remaining concentrations). The minimum objective function value (OFV) calculated by NONMEM (−2 log likelihood) was used for model discrimination between nested models. A dOFV of 3.84 was considered to be a significant difference between 2 nested models with a difference of one degree of freedom (p<0.05). Model assessment was based on successful minimization, OFV, the proper running of the covariance step, relative standard error of the parameter estimates, the goodness of fit plots and eta-shrinkage. Model validation was done using visual predictive checks and a bootstrapping procedure. The predictive performance of the models was evaluated using prediction corrected visual predictive checks (n=1000). The robustness of the final parameter estimates and the stability of the final model were validated using bootstrapping by generating 1000 resampled datasets with replacement.

Structural Models

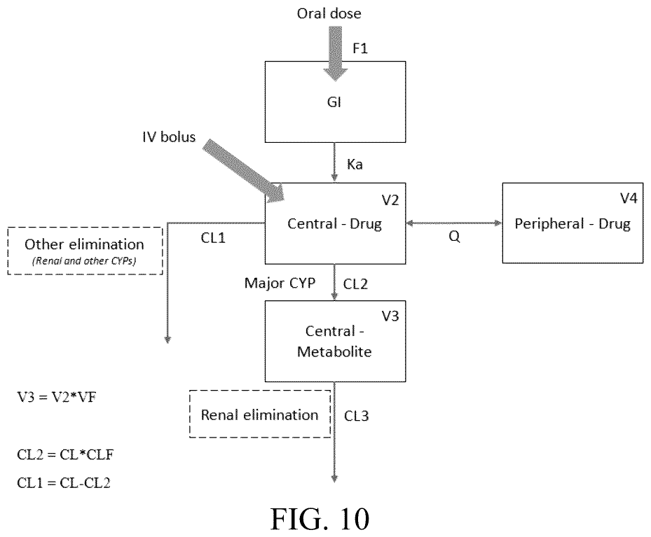

The model building process was done in a step-wise process. Multiple disposition models (one-, two- and three-compartment) were evaluated. Absorption models with various absorption parameters (first order absorption with and without lag, transit compartment models) were tried. Bioavailability was estimated as each patient had both intravenous and oral dose data. Interindividual variability was added to the model using an exponential error model (a log-normal distribution of eta with mean zero and variance ω². Residual unknown variability was described by a proportional error model (normal distribution of epsilon with mean zero and a variance of Σ²). The PK model for the drug was first built and the metabolite model was later added to it. The metabolite parameters were estimated considering the metabolite as a separate PK compartment. A depiction of a structural model for both R- and S-methadone are shown in FIG. 10, together with the estimated parameters for the model. In this model, V3 was parameterized as V2*Vf. Vf was the scaling factor between central volume of distribution of the drug and the metabolite. Vf was fixed to 1 in the model due to unidentifiability of the metabolite parameters as there were no urine data available for either the drug or the metabolite. CLF is the fraction of CL, the total clearance of the drug that contributes to CL2, the clearance towards the formation of the metabolite.

Covariate Models

All covariates were checked for biological plausibility and visualized graphically before being tested as a covariate for a specific parameter estimate. Univariate analysis of plausible pharmacokinetic parameters (the individual posthoc parameter estimates of those obtained from a model without covariates), with specific genotype, were done to reduce the number of testing in the covariate models. With the final list of covariate-parameter relationships, covariate selection was done using a step-wise covariate approach with a forward selection (P=0.05) and a more stringent backward elimination step (P=0.01).

The demographic covariates checked were biological gender, body weight, fat free mass, BMI and race. The physiological covariate checked was AAG concentration. AAG was measured at every time-point the concentrations were measured and were therefore used as a time-varying covariate in the model. Relationship with AAG was tested in accordance with equation (1):

Pₚₒₚ=Θₚ*[1±Θ_{AAG}*(AAG−AAG_{median})] (1)

where Θₚ is the typical value in the population, AAG is the concentration of AAG at a particular time point, Θ_{AAG} is the proportional factor for the change in the parameter estimate with a unit change in the concentration of AAG from the median concentration of AAG, AAG_{median}.

Genotypes were used as covariates with 2 distinct methods depending on the availability of allelic phenotypic information. Genotypic variants where allelic phenotype information was available were given an activity score depending on the previous literature description of the allelic variant, as follows. CYP2B*6*6, considered as a poor metabolizer, was given a score of 0; *1*1, considered as a normal metabolizer, was given a score of 1; and *1*6, considered as an intermediate metabolizer, was given a score of 0.5. The relationship between the pharmacokinetic parameter and the activity score of the CYP enzymes was checked using a linear relationship, a power relationship, and an exponential relationship, in accordance with equations (2), (3), and (4), respectively:

CL=CL₀*(1+Θ₁*(n−1)) (2)

where CL₀ is the clearance in an individual with an activity score of 1, n is the activity score of the specific CYP enzyme in a particular individual and Θ₁ represents the fractional change per active allele.

CL=CL₀+n^{Θ1} (3)

CL=CL₀*exp(n*Θ₁) (4)

where n is the activity score of the specific CYP enzyme in a particular individual and Θ₁ represents the average contribution per active allele. Though all the three models gave similar results, the linear (“proportional”) relationship was used in the final model for the easy interpretation of the results.

Genotypic variants where allelic phenotype information was not available were analyzed using the dominant, additive or recessive models. The genetic models are defined as: wild-type=1, Heterozygous=1 and Variant=0 under dominant model; wild-type=2, Heterozygous=1 and Variant=0 under additive model; wild-type=1, Heterozygous=0 and Variant=0 under recessive model. The relationships were represented by equation (2) in additive models and equation (5) in the dominant and recessive model.

CL=CL₀*(1+Θ₁*(n−1)) (2)

where CL₀ is the clearance in an individual with single active allele and Θ₁ represents the average contribution per active allele.

CL=Θ₁*n+Θ₂*(n−1) (5)

where Θ₁ represents the average clearance for allele coded 1 and Θ₂ represents the average clearance for allele coded 0 in both the dominant and recessive models.

### Results

Patients

A total of 61 children and adolescents aged 11-17 years participated in the present embodiment. The demographic details are given in Table 4.

TABLE 4
Demographics

| | Median (IQR) N = 61 | |
|---|---|---|
| Age (years) | 14.74 | (13.62-15.66) |
| Weight (kg) | 53.60 | (47.90-60.10) |
| Height (cm) | 164.50 | (158.00-171.50) |
| BMI | 19.40 | (17.61-22.50) |
| Baseline AAG | 84.00 | (61.92-108.88) |
| Biological sex | Females 31, Males = 30 | |
| Race | | |

| Caucasian | 49 |
|---|---|
| African American | 7 |
| Hispanic | 3 |
| Native American | 1 |
| Unknown | 1 |
| Type of surgery | Pectus excavatum repair = 25 |
| | Idiopathic scoliosis spinal fusion surgery = 36 |

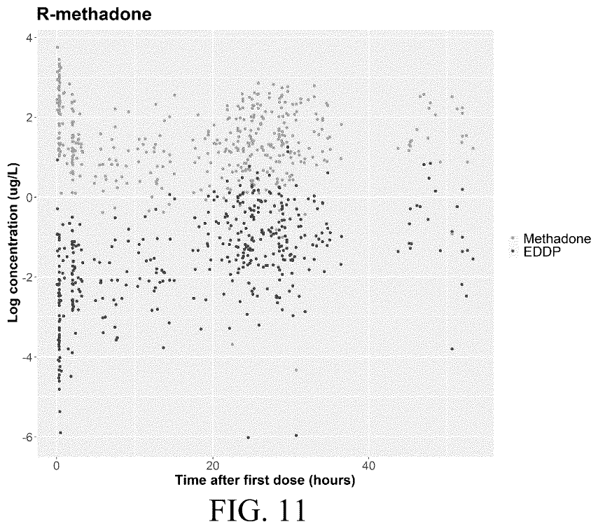

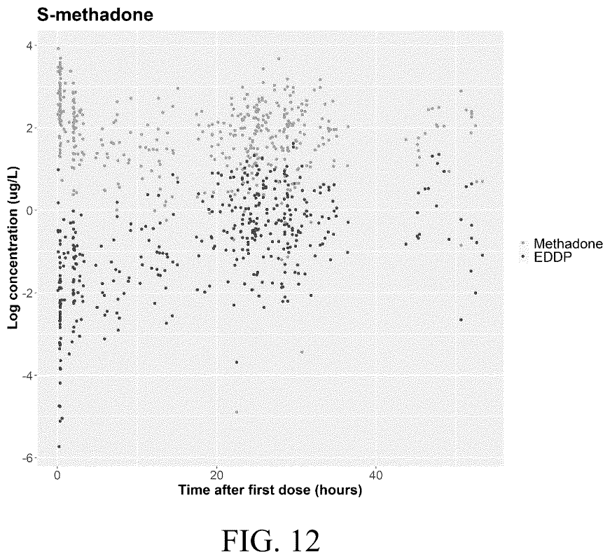

0.46% of the drug concentrations and 9.4% of the metabolite concentrations were below the lower limit of quantification. There were 430 drug and 381 metabolite concentrations available for the entire analysis. 8 children had missing covariate information. The concentration time profiles of R- and S-methadone are shown in FIGS. 11 and 12.

Structural Model

R-methadone concentration-time data were best described by a two-compartment disposition model. Interindividual variability was added only to CL, Vd of the central compartment V2 and the peripheral compartment V4. The absorption phase could be well described by a first order process estimated with unique absorption rate constants for the tablet and the suspension formulations. Absolute bioavailability was estimated without the estimation of its inter-individual variability. Addition of lag time or use of a transit compartment model did not improve the model fit (dOFV=1.376) or the RSE of the estimates. The metabolite model was added to the drug model after the drug model was finalized. To estimate the parameters of the metabolite, the drug parameters as derived from the previous step were fixed to the final estimates. The volume of distribution of the metabolite was not identifiable and therefore was set numerically equal to central volume of distribution of the drug (scaled by a factor named Vf fixed to 1). The metabolite concentrations were well described by a one-compartment model. Addition of a peripheral compartment did not improve the model fit.

S-methadone modeling was very similar to that of R-methadone, albeit with different parameter estimates.

Covariate Modeling on R Methadone

The covariates relationships checked for R-methadone are given in Table 5.

TABLE 5
List of covariates which were positive in base model (p < 0.05) but turned
insignificant (p > 0.01) with addition of other covariates

| | | | | | |
|---|---|---|---|---|---|
| | | | | dOFV from | Remarks if |
| SNP | Gene | PK parameter | Gene model | final | any |

R-methadone:

| | | | | | |
|---|---|---|---|---|---|
| rs7250601 | CYP2B6 | CLF | Recessive | −1.375 | |
| rs4646437 | CYP3A4 | CL | Recessive | −3.635 | |
| rs1042194 | CYP2C18 | CL3 | Additive | −4.285 | |
| rs2229109 | ABCB1 | CL3 | Recessive | −10.2 | Too few |
| | | | | | variants |
| Activity score | CYP2C19 | CL3 | Additive | −6.279 | |
| of CYP2C19 | | | | | |

S-methadone:

| | | | | | |
|---|---|---|---|---|---|
| rs3003596 | CAR (NR13) | CLF | Dominant | −5.95 | |
| rs2246709 | CYP3A4 | CLF | Additive | −3.651 | |
| rs1080983 | CYP2D6 | CLF | Additive | −0.016 | |
| rs17180299 | Unknown | CL3 | Recessive | −3.98 | Too few |
| | | | | | variants |
| rs1042194 | CYP2C18 | CL3 | Recessive | −7.93 | Increased the |
| | | | | | RSE on CL3 |
| | | | | | very much |
| Activity score | CYP2C19 | CL3 | Additive | −4.194 | |
| of CYP2C19 | | | | | |

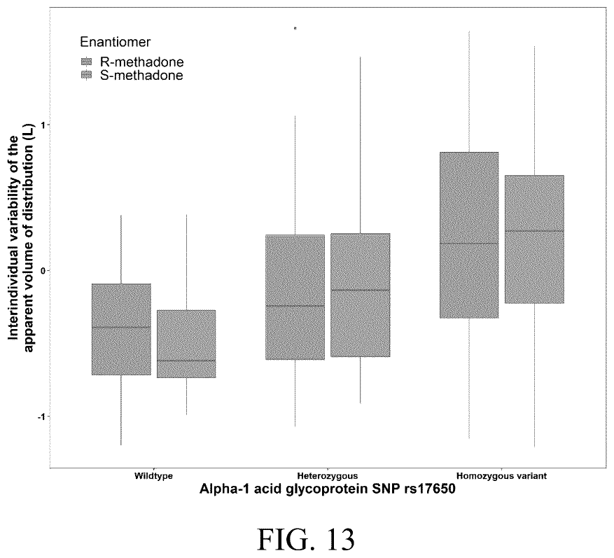

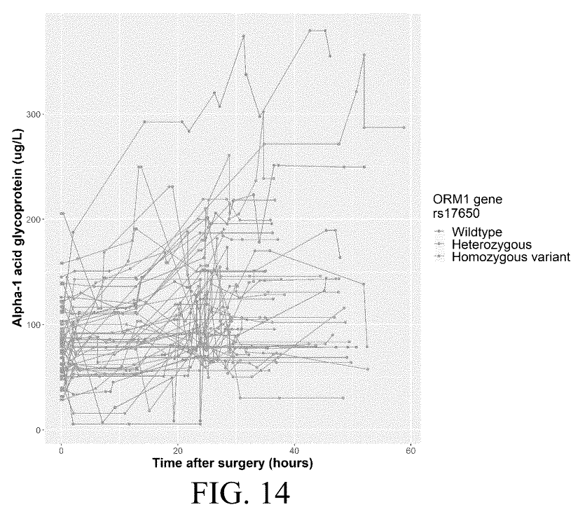

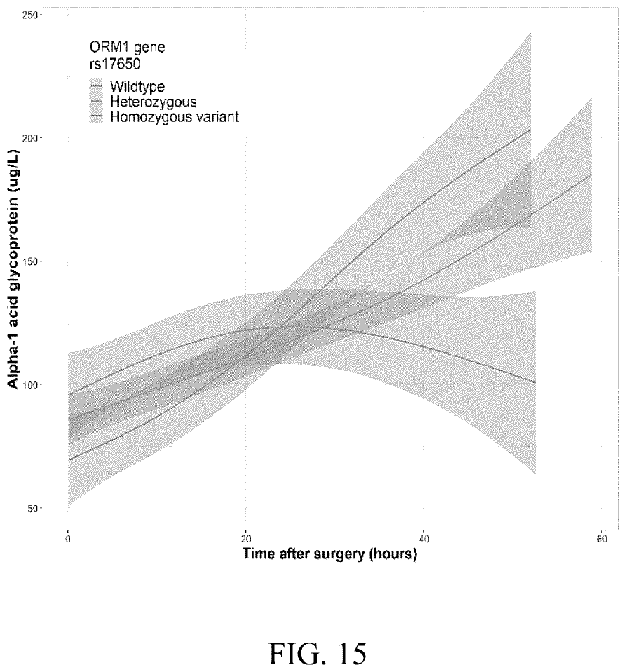

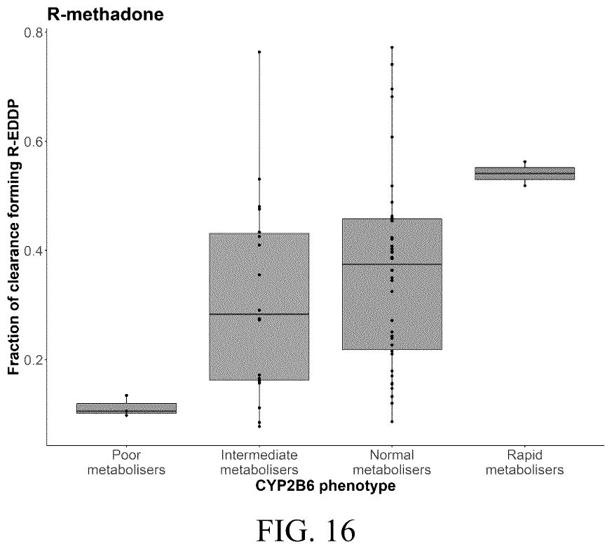

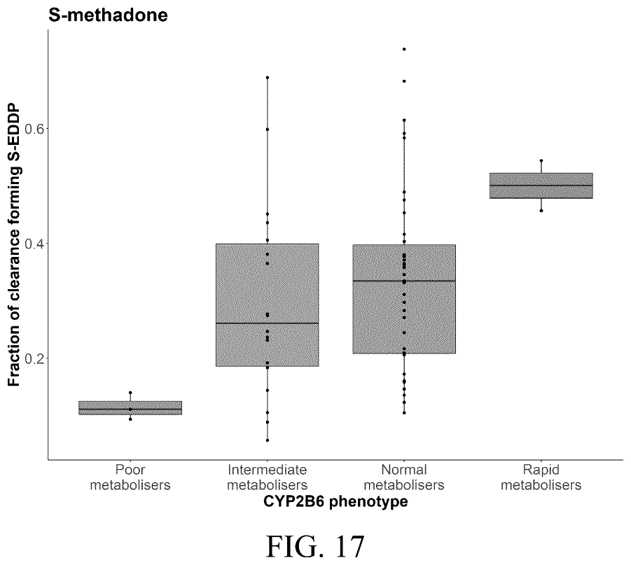

Body weight was added allometrically to the clearance of the drug with a fixed exponent of 0.75. A similar exercise on the intercompartmental clearance increased the RSE of the parameter. Also, allometric scaling of the volume parameters with body weight worsened the model fit as evidenced by an increase in the OFV (dOFV=1.2). Similar results were obtained with fat free mass. AAG concentration was a significant covariate on the volume of distribution of the central compartment V2 (dOFV=−7.2). AAG was also analyzed using the method 2 (described in methods). This was not implemented as the RSE of the estimated ODAAG and the improvement compared to method 1 was marginal (dOFV=−3.3) despite the addition of a parameter to describe the intraindividual variability. A SNP of ORM1 (gene encoding for AAG) rs17650 was found to affect the V2 in an additive gene model (dOFV=−3.4) in addition to the just described effect of AAG concentration. Also, the concentrations of AAG did not show any trend with the genotype of rs17650, as shown in FIG. 13 (Interindividual variability in the apparent volume of distribution (V2) of R- and S-methadone vs rs17650 after adding AAG concentration as a covariate on V2 showing that the rs17650 genotype seem to suggest a good relationship with the interindividual variability of V2 of R- and S-methadone) and FIG. 14 (Concentration of AAG in the postoperative period showing that the concentrations of AAG increased in most of the individuals following the surgery). In FIG. 14, the genotype rs17650 does not seem to be related to the baseline AAG concentrations or the increase in the concentrations over the post-operative period). However, addition of rs17650 as a covariate in methadone PK modeling further improved the fit in which AAG level was already a covariate, as shown in FIG. 15, which is a graph illustrating different patterns in postoperative AAG levels in patients with different AAG genetic variants (ORM1 rs17650). The difference in OFV in the addition of rs17650 was not enough to be considered significant in a stepwise approach, however it was retained for the final model in view of its strong biological plausibility and the significance of the same relationship in the covariate model of S-methadone. CYP2B6 phenotype (activity score) was found to have a linear relationship with the fraction of clearance of the drug towards the formation of the metabolite CLF (dOFV=−8.8). The relationship between CYP2B6 phenotype (activity score) and CL2 of R- and S methadone is graphically shown in FIGS. 16 and 17, which show that the phenotype seem to suggest a good relationship with the clearance of R- and S-methadone in the formation of their corresponding primary metabolites R- and S-EDDP. Addition of rs2246709 a SNP of CYP3A4 as a covariate on CLF further improved the model (dOFV=−7.2). Biological sex, age, BMI and race did not show any trend with the PK parameters and therefore were not tested for covariate relationship. None of the other relationships which were earlier found to be significant on univariate analysis (Table 6) were significant when added as a covariate in the final pharmacokinetic model. In TABLE 6, the p-values were not corrected for multiple testing.

TABLE 6
List of all the covariates with significant relationship
to PK parameters in a univariate regression analysis.

| | | | PK | Differ- | P- | Wild- | Hetero- | Homo- |
|---|---|---|---|---|---|---|---|---|
| Relation | Gene | Gene | parameter | ence | value | type | zygous | zygous |
| Additive | rs2229109 | ABCB1 | ETASCL3 | −0.301 | 0.004 | 50 | 2 | 0 |
| Recessive | rs2229109 | ABCB1 | ETASCL3 | −0.301 | 0.004 | 50 | 2 | 0 |
| Additive | rs2229109 | ABCB1 | RCL3 | −17.331 | 0.007 | 50 | 2 | 0 |
| Recessive | rs2229109 | ABCB1 | RCL3 | −17.331 | 0.007 | 50 | 2 | 0 |
| Additive | rs2229109 | ABCB1 | SCL3 | −2.036 | 0.011 | 50 | 2 | 0 |
| Recessive | rs2229109 | ABCB1 | SCL3 | −2.036 | 0.011 | 50 | 2 | 0 |
| Additive | rs2229109 | ABCB1 | SCLF | 0.096 | 0.019 | 50 | 2 | 0 |
| Recessive | rs2229109 | ABCB1 | SCLF | 0.096 | 0.019 | 50 | 2 | 0 |
| Dominant | rs2501873 | CAR (NR1\|3) | ETASCL3 | −0.147 | 0.006 | 15 | 28 | 9 |
| Dominant | rs3003596 | CAR (NR1\|3) | ETASCL3 | −0.147 | 0.006 | 16 | 27 | 9 |
| Dominant | rs2501873 | CAR (NR1\|3) | ETASCLF | 0.367 | 0.041 | 15 | 28 | 9 |
| Dominant | rs3003596 | CAR (NR1\|3) | ETASCLF | 0.367 | 0.041 | 16 | 27 | 9 |
| Additive | rs2502815 | CAR (NR1\|3) | RCL | 9.548 | 0.043 | 28 | 21 | 3 |
| Dominant | rs2501873 | CAR (NR1\|3) | SCL3 | −1.072 | 0.008 | 15 | 28 | 9 |
| Dominant | rs3003596 | CAR (NR1\|3) | SCL3 | −1.072 | 0.008 | 16 | 27 | 9 |
| Dominant | rs2501873 | CAR (NR1\|3) | SCLF | 0.05 | 0.016 | 15 | 28 | 9 |
| Dominant | rs3003596 | CAR (NR1\|3) | SCLF | 0.05 | 0.016 | 16 | 27 | 9 |
| Dominant | rs10500282 | CYB2B6 | ETASCL | −0.617 | 0.043 | 34 | 17 | 1 |
| Dominant | rs10853744 | CYB2B6 | ETASCL | −0.617 | 0.043 | 34 | 17 | 1 |
| Dominant | rs3745274 | CYB2B6 | ETASCL | −0.617 | 0.043 | 36 | 15 | 1 |
| Dominant | rs8192719 | CYB2B6 | ETASCL | −0.617 | 0.043 | 36 | 15 | 1 |
| Dominant | rs10403955 | CYB2B6 | ETASCL | −0.427 | 0.05 | 35 | 15 | 2 |
| Additive | rs11882424 | CYB2B6 | ETASCL3 | −0.14 | 0.045 | 47 | 5 | 0 |
| Recessive | rs11882424 | CYB2B6 | ETASCL3 | −0.14 | 0.045 | 47 | 5 | 0 |
| Additive | rs11882424 | CYB2B6 | ETASCLF | 0.604 | 0.008 | 47 | 5 | 0 |
| Recessive | rs11882424 | CYB2B6 | ETASCLF | 0.604 | 0.008 | 47 | 5 | 0 |
| Recessive | rs3745274 | CYB2B6 | RCLF | −0.091 | 0.001 | 36 | 15 | 1 |
| Recessive | rs8192719 | CYB2B6 | RCLF | −0.091 | 0.001 | 36 | 15 | 1 |
| Additive | rs3745274 | CYB2B6 | RCLF | −0.087 | 0.003 | 36 | 15 | 1 |
| Additive | rs8192719 | CYB2B6 | RCLF | −0.087 | 0.003 | 36 | 15 | 1 |
| Recessive | rs10403955 | CYB2B6 | RCLF | −0.077 | 0.007 | 35 | 15 | 2 |
| Recessive | rs10500282 | CYB2B6 | RCLF | −0.071 | 0.011 | 34 | 17 | 1 |
| Additive | rs10500282 | CYB2B6 | RCLF | −0.066 | 0.021 | 34 | 17 | 1 |
| Additive | rs10403955 | CYB2B6 | RCLF | −0.067 | 0.022 | 35 | 15 | 2 |
| Recessive | rs10853744 | CYB2B6 | RCLF | −0.06 | 0.037 | 34 | 17 | 1 |
| Additive | rs7250601 | CYB2B6 | RCLF | −0.062 | 0.037 | 34 | 16 | 2 |
| Recessive | rs7250601 | CYB2B6 | RCLF | −0.06 | 0.037 | 34 | 16 | 2 |
| Additive | rs7250991 | CYB2B6 | RCLF | −0.062 | 0.037 | 34 | 16 | 2 |
| Recessive | rs7250991 | CYB2B6 | RCLF | −0.06 | 0.037 | 34 | 16 | 2 |
| Additive | rs11882424 | CYB2B6 | SCLF | 0.057 | 0.032 | 47 | 5 | 0 |
| Recessive | rs11882424 | CYB2B6 | SCLF | 0.057 | 0.032 | 47 | 5 | 0 |
| Additive | rs1042194 | CYP2C18 | ETARCL3 | −0.235 | 0.048 | 41 | 10 | 1 |
| Additive | rs12777823 | CYP2C18 | ETARCL3 | −0.235 | 0.048 | 41 | 10 | 1 |
| Additive | rs1042194 | CYP2C18 | ETASCL3 | −0.129 | 0.014 | 41 | 10 | 1 |
| Additive | rs12777823 | CYP2C18 | ETASCL3 | −0.129 | 0.014 | 41 | 10 | 1 |
| Recessive | rs1042194 | CYP2C18 | ETASCL3 | −0.121 | 0.015 | 41 | 10 | 1 |
| Recessive | rs12777823 | CYP2C18 | ETASCL3 | −0.121 | 0.015 | 41 | 10 | 1 |
| Additive | rs1042194 | CYP2C18 | SCL3 | −0.927 | 0.02 | 41 | 10 | 1 |
| Recessive | rs1042194 | CYP2C18 | SCL3 | −0.881 | 0.02 | 41 | 10 | 1 |
| Additive | rs12777823 | CYP2C18 | SCL3 | −0.927 | 0.02 | 41 | 10 | 1 |
| Recessive | rs12777823 | CYP2C18 | SCL3 | −0.881 | 0.02 | 41 | 10 | 1 |
| Additive | rs28360521 | CYP2D6 | ETASCL | −0.209 | 0.014 | 28 | 22 | 2 |
| Recessive | rs28360521 | CYP2D6 | ETASCL | −0.188 | 0.024 | 28 | 22 | 2 |
| Recessive | rs1080983 | CYP2D6 | ETASCL3 | −0.168 | 0.015 | 5 | 21 | 26 |
| Additive | rs1080983 | CYP2D6 | ETASCL3 | −0.152 | 0.037 | 5 | 21 | 26 |
| Additive | rs1080983 | CYP2D6 | ETASCLF | 0.672 | 0.005 | 5 | 21 | 26 |
| Recessive | rs1080983 | CYP2D6 | ETASCLF | 0.622 | 0.006 | 5 | 21 | 26 |
| Additive | rs28360521 | CYP2D6 | SCL | −2.221 | 0.035 | 28 | 22 | 2 |
| Recessive | rs1080983 | CYP2D6 | SCL3 | −1.281 | 0.014 | 5 | 21 | 26 |
| Additive | rs1080983 | CYP2D6 | SCL3 | −1.188 | 0.032 | 5 | 21 | 26 |
| Recessive | rs2246709 | CYP3A4 | ETARCLF | −0.312 | 0.028 | 23 | 24 | 5 |
| Additive | rs3735451 | CYP3A4 | RCL | 16.254 | 0.006 | 39 | 9 | 4 |
| Recessive | rs3735451 | CYP3A4 | RCL | 13.005 | 0.011 | 39 | 9 | 4 |
| Additive | rs2242480 | CYP3A4 | RCL | 14.374 | 0.012 | 38 | 10 | 4 |
| Recessive | rs2242480 | CYP3A4 | RCL | 11.879 | 0.018 | 38 | 10 | 4 |
| Recessive | rs4646437 | CYP3A4 | RCL | 11.343 | 0.025 | 38 | 11 | 3 |
| Additive | rs4646437 | CYP3A4 | RCL | 11.967 | 0.032 | 38 | 11 | 3 |
| Dominant | rs4646437 | CYP3A4 | RCL3 | 10.716 | 0.048 | 38 | 11 | 3 |
| Dominant | rs10211 | CYP3A7 | ETARCL | −0.35 | 0.049 | 3 | 10 | 39 |
| Dominant | rs10211 | CYP3A7 | RCL | −13.24 | 0.01 | 3 | 10 | 39 |
| Dominant | rs2257401 | CYP3A7 | RCL | −13.005 | 0.011 | 3 | 10 | 39 |
| Dominant | rs17650 | ORM1 | RCL | 9.741 | 0.05 | 7 | 30 | 15 |
| Dominant | rs2276707 | PXR (NR1\|2) | ETASCL | −0.484 | 0.025 | 32 | 18 | 2 |
| Additive | rs17180299 | rs17180299 | ETASCL3 | 0.176 | 0.046 | 49 | 3 | 0 |
| Recessive | rs17180299 | rs17180299 | ETASCL3 | 0.176 | 0.046 | 49 | 3 | 0 |
| Additive | rs17180299 | rs17180299 | ETASCL3 | 0.176 | 0.046 | 49 | 3 | 0 |
| Recessive | rs17180299 | rs17180299 | ETASCL3 | 0.176 | 0.046 | 49 | 3 | 0 |
| Additive | rs17180299 | rs17180299 | SCL3 | 1.358 | 0.042 | 49 | 3 | 0 |
| Recessive | rs17180299 | rs17180299 | SCL3 | 1.358 | 0.042 | 49 | 3 | 0 |
| Additive | rs17180299 | rs17180299 | SCL3 | 1.358 | 0.042 | 49 | 3 | 0 |
| Recessive | rs17180299 | rs17180299 | SCL3 | 1.358 | 0.042 | 49 | 3 | 0 |

Phenotypes of alleles

| | | | PK | | | | |
|---|---|---|---|---|---|---|---|
| Gene | Difference | P-value | parameter | UM | NM | IM | PM |
| CYP2B6 | −0.176 | 0.001 | RCLF | 2 | 31 | 17 | 4 |
| CYP2B6 | −0.425 | 0.014 | ETASCL | 2 | 31 | 17 | 4 |
| CYP2B6 | −0.065 | 0.05 | SCLF | 2 | 31 | 17 | 4 |
| CYP2C19 | 0.073 | 0.039 | ETASCL3 | 17 | 26 | 10 | 1 |
| CYP2D6 | −7.063 | 0.005 | SCL | 1 | 10 | 34 | 9 |
| CYP2D6 | −0.569 | 0.006 | ETASCL | 1 | 10 | 34 | 9 |
| CYP2D6 | 0.699 | 0.046 | ETASCLF | 1 | 10 | 34 | 9 |
| CYP3A5 | 8.784 | 0.045 | RCL | 0 | 2 | 7 | 45 |

In Table 6, UM: Ultrarapid/rapid metabolizers, NM: Normal/Extensive metabolizers, IM: Intermediate metabolism, PM: Poor metabolizers. These were all the significant relationships seen between the various genotypes and the various clearance parameters.

Covariate Modeling on S Methadone

The covariates relationships checked for S-methadone are given in Table 5. Body weight was added allometrically to the clearance of the drug with a fixed exponent of 0.75. Allometric scaling of the intercompartmental clearance and distribution parameters of the drug with body weight or fat free mass worsened the model fit (dOFV=3.9). AAG was once again a significant covariate on V2 of the drug (dOFV=−6.3). Similar to R-methadone rs17650 was found to have a covariate relationship with V2, however with a better improvement in model fit (dOFV=−7.2). Again, CYP2B6 phenotype (activity score) was found to have a linear relationship with CLF (dOFV=−8.8). The intronic SNP rs1188424 further improves the model when used as a covariate on CLF (dOFV=−9.8). Alternative to the addition of rs11882424, the CYP3A4 SNP rs2246709 was again found to be significantly (dOFV=−8.9) related to CLF in the absence of rs11882424 in the model. No other covariate relationships were significant enough to be added to the model.

Model Qualification

The visual predictive checks indicate that the simulated concentrations of R- and S-methadone agree with the observed data. The 5th, 50^{th}, and 95^{th} percentiles of the observed data fall within the respective 95% prediction intervals of the simulated data. A bootstrapping procedure (n=1000) found the parameter estimates to be reliable with acceptable relative standard errors (Table 5). The η and the ε shrinkage were found to reasonably low.

The final model for R methadone has the following covariate relationships represented by equations (6) and (7).

CLF=0.217*(1+0.745*(CYP2B6 activity score −1))*(1+0.45*(number of active alleles rs2246709−1))*(Body weight/70)**0.75 (6)

where 0.217 is the CLF in a 70 kg individual with CYP2B6 activity score of 1 and a heterozygous rs2246709.

V2=176L*(1−0.443*(number of active alleles rs17650−1)*(1−0.00291*(AAG conc −94.76) (7)

where 176 L is the V2 in an individual with a heterozygous rs17650 and an average AAG concentration of 94.76 μg/mL.

The final model for S methadone has the following covariate relationships represented by equations (8), (9), and (10).

CLF=0.135*(1+0.636*(CYP2B6 activity score −1))*(1+1.68[if recessive rs11882424])*(Body weight/70)**0.75 (8)

where 0.135 is the CLF in a 70 kg individual with CYP2B6 activity score of 1 and has a dominant rs11882424 genotype. Another model was also considered in parallel because of the questionable plausibility of rs11882424 effects on clearance.

CLF=0.128*(1+0.553*(CYP2B6 activity score −1))*(1+0.358*(number of active alleles rs2246709−1))*(Body weight/70)**0.75 (9)

where 0.128 is the CLF in a 70 kg individual with CYP2B6 activity score of 1 and a heterozygous rs2246709.

V2=98.3L*(1−0.526*(number of active alleles rs17650−1)*(1−0.00192*(AAG conc −94.76) (10)

where 98.3 L is the V2 in an individual with a heterozygous rs17650 and an average AAG concentration of 94.76 μg/mL.

### Discussion

This Example demonstrated that the concentration of AAG and the ORM1 genotype independently affect the volume of distribution of both the R- and S-enantiomers of methadone. CYP2b6 genotype and other new intronic genotypes also contribute to the variability in the clearance of the enantiomers.

The R- and the S-enantiomers of methadone are different in their pharmacodynamic and pharmacokinetic properties. The R enantiomer is mainly responsible for analgesia, sedation and respiratory depression while the S enantiomer is responsible for its QT prolongation. There is a lot of interindividual variability in the exposure of methadone owing primarily to the involvement of multiple cytochrome P (CYP) enzymes in its metabolism and its binding to the acute phase protein AAG in the plasma. Methadone is mainly oxidized by various CYP enzymes in the liver. R methadone is metabolized mainly by CYP3A, CYP2B6, CYP2C19 and CYP2C8 in order of importance. S methadone is metabolized mainly by CYP3A, CYP2B6, CYP2D6 and CYP2C18 in order of importance. Other CYP enzymes involved in the metabolism do not contribute much in this regard. Other genetic determinants of variability implicated in the metabolism of the drug are ABCB1 (P-glycoprotein transporter) and POR (Cytochrome P450 reductase). 2-Ethylidene-1,5-dimethyl-3,3-diphenylpyrrolidine (EDDP) is the primary metabolite of the drug and is known to be primarily inert except for a possible association with QT prolongation. Methadone is bound 80-90% to AAG in the plasma. The concentrations of AAG being an acute phase protein can increase many folds during stressful conditions like inflammation, infections, burns and surgeries. This can affect the effective exposure of the drug by reducing its unbound concentrations. The orosomucoid genes ORM1 and ORM2 encode for AAG. Although there is some data available on the binding of the drug to AAG, the effect of its genetic polymorphism on the binding of the drug is unknown.

A pharmacokinetic modelling approach described here enables exploration of the sources of variability in the pharmacokinetics of the drug and its major metabolite EDDP in a pediatric population undergoing major surgeries. R- and S-methadone were well described by a two-compartment disposition model with first order absorption and elimination. The corresponding metabolites were described well by one compartment disposition models with first order elimination. Clearance of both R- and S-methadone were allometrically scaled by bodyweight. CYP2B6 phenotype was a determinant of the clearance of both the enantiomers in an additive gene model. rs2246709, an intronic SNP on CYP3A4, was found to decrease the clearance of R- and S-methadone. AAG and the SNP of AAG rs17650 independently influenced the volume of distribution of both the enantiomers.

Population pharmacokinetic modeling was used in this Example to study the variability of pharmacokinetics of R- and S-methadone and study the effect of various known covariates on it. The use in the Example of both intravenous and oral doses, either tablet or suspension, depending on the patient's convenience helped in the estimation of absolute bioavailability and the estimation of accurate estimates of the PK parameters without the effect of relative bioavailability on them. The BLOQ concentrations were omitted according to the M1 method due to the relatively small numbers of BLOQ data. The BLOQ values were mainly the samples towards the end of the concentration curve in case of the drug, and towards the beginning of the curve in the case of the metabolites. Although, unique absorption rate constant could be estimated for the two oral formulations, individual bioavailability terms could not be estimated. Also, introduction of absorption lag time or a transit compartment absorption model did not improve the model. Without intending to be bound by any particular theory, this was probably due to the sparsity of concentrations in the absorption phase and the use of two different oral formulations in the data adding to the variability. Allometric weight scaling was done on the clearance of both R- and S-methadone. Scaling of other parameters was not done as they were found to worsen the model fit or increase the uncertainty of the parameter estimates. Estimation of scaling factor improved the fit, yet the factor was fixed to a standard value of 0.75 to prevent false estimation or bias in the estimation of scaling factor with a smaller sample size.

The major binding protein AAG being an acute phase protein increased in most patients in the post-surgical period. The median baseline AAG concentration was 84 μg/mL and varied between 24.48 to 205.38 μg/mL in the population. This variability increased through the period of in-hospital stay in most patients, as shown in FIG. 14 and as noticed in similar studies in the past. This was captured in the time-varying covariate relationship between the volume of distribution of the drug V2 and AAG.

The AAG is encoded by two loci, ORM1 and ORM2. Without intending to be bound by any particular theory, it is believed that ORM2 is widely preserved and is not polymorphic, while ORM1 can be polymorphic. Rs17650 is a SNP which may determine the phenotype of AAG as F and S, and rs1126801 further classifies F as F1 and F2. The variant F phenotypes have lesser binding for methadone than the S phenotype. Accordingly, an interesting finding is the relationship between rs17650 and V2 in the Example. While this was prominently seen with S-methadone (dOFV=−7.2), it was not so prominent with R-methadone (dOFV=−3.4). As both R- and S methadone were shown to be bound to AAG and in the absence of evidence that R- and S-methadone have differential binding to AAG or its subtypes, rs17650 was considered as a covariate on V2 of R-methadone due to the observed significant effect on the V2 of S-methadone. The other SNP rs1126801 was poorly represented in the study population (n=2) and therefore was not used for further analysis.

CYP enzymes including CYP3A4, CYP2B6, CYP2C19, CYP2C8, CYP2D6 and CYP2C18 have been shown in the past to be involved in the metabolism of methadone with varying results in-vitro. Although CYP3A4 contributes to more than 50% of the metabolism of the drug, it is believed that CYP3A4 possesses less genotypic variability and, therefore, may be considered to have less impact on the variability of the metabolism of methadone to its primary metabolite. Out of all the CYP enzymes, CYP2B6 has been consistently shown to most contribute to the variability in the metabolism of the drug. Due to the vast number of CYP enzymes involved in the metabolism of methadone, it is difficult to isolate the effect of an individual enzyme on clearance in-vivo. Some of the genotypes in the CYPs are well documented and have a known phenotypic activity, while other genotypes are just known as individual SNPs and can be related to the clearance of the drug by any of the gene models such as additive, dominant or recessive. This resulted in a wide number of phenotypes and genotypes to be tested as covariates on the clearances of the drug and its metabolite. Therefore, a two-staged process was employed in the selection of the covariates. Initially a univariate analysis was done between the biologically plausible covariates and the parameter estimates. The parameter-covariate pairs found to have a significant relationship (p<0.05) were used in the covariate model building exercise. Relationships with negligible actual difference in mean parameter estimates between the groups or with genotypes with underrepresented variant population were not used for covariate modelling. CYP2B6, CYP2D6, CYP2C19, CYP3A4 and CYP3A5 were tested using their activity scores as these alleles are well studied. Most of the activity scores were not found to be related to the various clearances of R- and S-methadone. CYP2B6 was found to be strongly related to the metabolism of the parent drug towards the primary metabolite EDDP in a significant manner for both the enantiomers. rs11882424 is an intronic SNP of CYP2B6 and was earlier found to affect the clearance of methadone although the direction of influence is unclear. In the present Example, this SNP was found to be highly significant (dOFV=−9.8) in relation to CLF of S-methadone. The variant allele of the same was found to increase the CLF of S-methadone, which has not been reported in literature elsewhere in the past. In order to maintain a conservative approach, rs11882424 was excluded from the model and included rs2246709 (dOFV=8.9), the next in line significant SNP.

CYP2C19 activity scores were associated with the clearance of R- and S-EDDP but were not retained to the final model because of the use of stricter backward elimination step and a marginal increase in RSE of the clearance of the metabolite CL3. CYP3A4 and CYP3A5 activity scores were tested individually and as a cumulative score and were not found to be significant. Though CYP3A4 has a significant role in the metabolism of the drug, it is generally less polymorphic and had a low frequency of heterozygous*22(n=6) and no homozygous*22 in the study population. Interestingly one of the intronic SNPs of CYP3A4 rs2246709 was found to be a better predictor of R-methadone CLF and was therefore retained in the model. CYP2C18 has been shown previously in in-vitro studies to metabolize R- and S-methadone. Its SNP rs1042194 seems to have an effect on CL3, the clearance of both R- and S-EDDP.

rs2229109 (1199G>A) a possibly relevant variant of ABCB1 when added as an covariate on CL3 of R- and S-methadone showed an improvement in OFV, but had so few individuals with the variant that it was dropped from the final model (n=2). Similar decision was taken with rs17180299, a variant found to influence the clearance in a GWAS study. The NR1I subfamily receptors, the constitutive active/androstane receptor (CAR) and the pregnane λ receptor (PXR) regulates numerous metabolic enzymes including the CYP enzymes studied. rs3003596 a SNP on CAR shows a trend in relation with CLF of S-methadone.

The present work has a small list of significant covariates added to the final model compared to the exhaustive list of CYPs tested. This is due to the relatively smaller sample size and the lack of variability seen with certain genotypes in the studied population. Moreover, most of the other studies with multiple significant CYPs were univariate analysis comparing genotype with point concentrations like trough or Cₘₐₓ. Prior PK modeling work has used a lower threshold for the backward elimination to retain the covariates in the model. Earlier in the present Example rs17650 was used as a covariate on AAG despite p>0.01 citing strong biological plausibility. Yet a stricter approach was used in the case of covariates related to clearances. This decision was made in the beginning of the present work to have a stricter backward elimination step especially in relation to various clearances in view of the multitude of covariate-parameter relationships. Many of these relationships could become significant, however, with a larger population, where the effects of multiple CYPs could be meaningfully separated. All the relationships that were significant from the univariate analysis and the covariate modelling are set forth in Tables 5 and 6.

In conclusion, this was an extensive analysis of genomic data and pharmacokinetic data in children undergoing major surgeries. Multiple covariates in different gene models affecting the volume of distribution and the various clearances of the drug were analyzed and non-linear mixed effects models were used to identify appropriate covariates describing adequately the pharmacokinetics of R- and S-methadone. Concentration of AAG and the ORM1 genotype independently affect the volume of distribution of both the enantiomers. CYP2B6 genotype and other intronic genotypes contribute to the variability in the clearance of the enantiomers. Knowledge of the effect of genotype on the pharmacokinetics can be used to achieve more precise dosing of enantiomeric methadone for analgesia.

### Example 3

### Association Between CYP2B6 Polymorphisms and Perioperative Methadone Metabolism

Genetic polymorphisms influencing methadone pharmacokinetics and pharmacodynamics contribute to the significant variability in responses to methadone administration in terms of analgesic efficacy and adverse events. Methadone is a semisynthetic opioid agonist, with additional action on N-Methyl-D-aspartate (NMDA) and serotonin-norepinephrine reuptake Inhibitor (SNRI) pathways. It is long acting and has been long used in the management of opioid use disorders (OUD). Along with the long duration of action, methadone is promising in terms of protection against chronic post-surgical pain, minimal abuse potential, minimal risk of opioid sensitization and opioid induced hyperalgesia. The analgesic potential of methadone is being increasingly recognized in the perioperative environment, both in adults and children, over the past two decades. The main barrier for widespread implementation has been risk of life-threatening respiratory depression and deaths. Additionally, methadone dosing strategy for perioperative use has largely been speculative, typically given in the form of a single large dose administered intra-operatively.

Methadone elimination is primarily hepatic, via the CYP enzymes. Though a number of CYP enzymes including CYP2B6, CYP2D6, CYP2C19, CYP2C9 and CYP3A4 have been described in methadone metabolism, CYP2B6 seems to be the most significant enzyme mediating stereoselective N-demethylation of both R- and S-methadone enantiomers to their major and a number of minor metabolites, which are all inactive. CYP2B6 expression shows 20- to 250-fold variability. Variants in CYP2B6 have been shown to significantly impact methadone PK in adults. CYP2B6 *6/*6 was associated with 45% lower clearance in S-methadone and 30% lower clearance in R-methadone compared with the wild type, *1/*1, in 64 healthy adults taking a single methadone dose.

Adult studies have associated CYP2B6 polymorphism with methadone response and dose requirements in the setting of methadone maintenance therapy (MMT) for OUD. Understanding the role of CYP2B6 genetic variants in inter-individual variability in perioperative methadone's metabolism, analgesic and adverse clinical effects is essential to formulate personalized safe and effective surgical pain management and precision dosing.

This Example explores the influence of CYP2B6 polymorphism on perioperative methadone's metabolism, analgesia and clinical outcomes such as methadone induced post-operative nausea and vomiting (PONY) in children and adolescents.

### Methods

This Example is a prospective, genotype-blinded, clinical observational study of children undergoing Nuss bar repair for pectus excavatum (PE) and posterior spinal fusion (PSF) repair for idiopathic scoliosis that received multidose methadone-based multimodal analgesia, performed in a tertiary care pediatric hospital. It aimed to study possible associations between CYP2B6 genetic variants, methadone pharmacokinetics and clinical safety and efficacy outcomes of perioperative methadone.

Study Participants

All children aged 8 to 17.9 years old undergoing PE and PSF surgeries were eligible for this study. Exclusion criteria include the following: allergy to methadone, developmental delay, neurological disorder, liver and/or renal disease, and preoperative pain requiring analgesics.

Analgesia Protocol

Multimodal analgesia protocols as illustrated in TABLE 1 set forth above in Example 1 were followed for all the participants undergoing the surgeries.

Specimen Collection, Storage, and Analysis

Blood samples were collected at specific time windows, including at 10-30 minutes, 2-6 hours, 10-12 hours after dose 1 and 3, 1-2 hours, and 10-12 hours after dose 2 through a designated intravenous line in situ for pharmacokinetic analysis, concentration-time profile of methadone and metabolites. Plasma levels of R- and S-methadone and primary metabolite: 2-Ethylidene-1,5-dimethyl-3,3-diphenylpyrrolidine (EDDP) were measured using high-performance liquid chromatography with tandem mass spectrometry (HPLC-MS/MS) chiral assay; Metabolite to parent ratios of R-, S- and total methadone were calculated. The baseline sample before methadone administration was obtained after intravenous (IV) catheter placement in the operative room (OR), this was used for genotyping. DNA was isolated on the same day and frozen at −20° C.

Genotyping

Genome sequencing analysis was done at 40× coverage (mapped to hg 19) to provide adequate read depth to cover all the relevant genetic variants of CYP2B6 and to accurately detect the copy numbers. Aldy1, a high-throughput sequencing data analysis tool with a combinatorial optimization framework to resolve allelic decomposition of highly polymorphic CYP2B6 gene was used to identify the genotypes from the mapped data. 16 previously studied common CYP2B6 single nucleotide polymorphisms (SNP), with minor allele frequency 5% were selected to be included in analysis. These included rs4803419, rs2279344, rs1038376, rs10403955, rs10500282, rs10853744, rs11882424, rs2279342, rs2279343, rs2279345, rs3745274, rs707265, rs7250601, rs7250991, rs8100458 and rs8192719. These SNPs were assessed for linkage disequilibrium (LD) and those in strong LD were deemed redundant and excluded from analyses. 8 SNPs were included in the final analyses (rs4803419, rs2279344, rs1038376, rs10500282, rs11882424, rs2279342, rs2279343, rs8100458).

Clinical Outcome Measures

The primary clinical outcome measured was PONV. The secondary outcome measured was maximum postoperative pain scores. Episodes of PONV were recorded as a binary outcome from nursing reports on electronic health records. Analgesic efficacy was described in terms of patient self-reported pain scores. Pain scores were collected every 2 to 4 h using a self-reported numeric rating scale ranging from 0 to 10.

Statistical Analysis

Analyses were performed using the dominant, additive or recessive models to explore for possible associations. Associations were tested using ‘A’ as the referent allele and ‘a’ as the alternative allele under dominant model (i.e. AA vs. Aa/aa), additive model (i.e. AA vs. Aa vs. aa) and recessive model (i.e. AA/Aa vs. aa). Linear regression was used to investigate SNP associations for pain score. Poisson regression was used to investigate SNP associations for PONV. Unadjusted as well as covariate-adjusted analyses, adjusted for weight, race, age, surgery type and sex were performed. For primary outcome, PONV, the SNP's rate ratios (i.e. exponential of the slope parameter in the Poisson regression), p-values and 95% Cis were computed. For postoperative pain score, the SNPs' effect (i.e. the slope parameter in the linear regression model), p-values, and 95% confidence intervals (CIs) were computed.

Methods for Metabolite/Methadone Area Under the ROC Curve (AUC) Ratio

The association under dominant model (i.e. AA vs. Aa/aa) with Metabolite/Methadone AUC Ratio was examined. Linear regression was used to investigate both R- and S-methadone metabolite/methadone AUC ratio. First, unadjusted analyses (i.e. simple linear regression) were conducted. Second, covariate-adjusted analyses (i.e. multiple linear regression) were conducted, which adjusted for weight, race, age, surgery and sex. In both analyses, the SNPs' slopes, p-values and 95% confidence intervals (CIs) of the slopes were computed.

Because eight SNPs were used for the final analysis, a Bonferroni correction was used for multiple comparisons for the primary outcome measure, which yielded a significance threshold of p=0.00625 [p=0.05/8 SNPs)]. Associations reaching threshold of 0.05 as nominally associated for secondary clinical outcome and metabolite/methadone AUC ratio are reported herein.

### Results

As discussed further below, CYP2B6 poor metabolizers (*6/*6) had >2-fold lower R- and S-methadone metabolites to methadone ratio compared with normal and rapid metabolizers. The incidence of PONV was 4.7× greater in those with CYP2B6 rs1038376 variant. AG/GG variants of rs2279343 SNP had 2.86-fold higher incidence of PONV compared to the wild variant (AA) in the unadjusted models. Nominal associations between variants of rs10500282, rs11882424 and rs4803419 and maximum postoperative pain scores were also observed.

Demographics

A total of 53 children were recruited. Mean age of the participants was 14 years, and majority of the participants were Caucasians (87%). About half of the participants were females. 45% of the participants underwent pectus repair and 55% underwent a posterior spine fusion (Table 7).

TABLE 7
Characteristics of Participants

| Variable | Count | Count (%) |
|---|---|---|
| Race | | |

| | |
|---|---|
| Caucasian | 46 (87%) |
| Other | 7 (13%) |

Surgery type

| | |
|---|---|
| Pectus repair | 24 (45%) |
| Spine fusion | 29 (55%) |

Biological gender

| | |
|---|---|
| Male | 27 (51%) |
| Female | 26 (49%) |

| Variable | Mean | Standard Deviation |
|---|---|---|
| Weight (kg) | 56.5 | 13.7 |
| Age (years) | 14 | 1.6 |
| Length of hospital stay (days) | 2.7 | 0.8 |
| Morphine equivalent (mg/kg) - Pectus | 0.7 | 0.2 |
| Morphine equivalent (mg/kg) - Fusion | 0.6 | 0.2 |

Phenotype Associations with PONV and Pain Scores

Among the 53 participants, 30 were normal/extensive metabolizers, 17 were intermediate metabolizers, 4 were poor metabolizers and 2 were rapid metabolizers. There did not appear to be any significant difference between the different metabolizing groups and clinical outcomes with just 2 rapid metabolizers and 4 poor metabolizers.

CYP2B6 Variants and Clinical Outcomes

Novel CYP2B6 genetic associations were identified between methadone-induced PONV and CYP2B6 SNPs, rs1038376 (P=0.005), rs10853744 (P=0.024), rs7250601 and rs7250991 (P=0.022), as well as post-surgical pain (rs11882424, P=0.007).

Genetic Associations with PONV (Table 8)

Significant association was found between rs1038376 variant and the incidence of PONV as shown in Table 8 below:

TABLE 8
CYP2B6 genetic associations with methadone induced nausea and vomiting,
adjusted for covariates including weight, race, age, surgery type and sex

| | | | | | | | | Covariate adjusted | | |
|---|---|---|---|---|---|---|---|---|---|---|
| | Referent | Minor | | | Unadjusted analysis | | | analysis | | |
| | allele | allele | Referent | Alternative | Rate | | | Rate | | |
| SNP | (Freq.) | (Freq.) | Group | group | ratio | P | 95% CI | ratio | P | 95% CI |

Dominant model

| | | | | | | | | | | |
|---|---|---|---|---|---|---|---|---|---|---|
| rs2279343 | A (0.94) | G (0.06) | AA | AG/GG | 2.68 | 0.025 | 1.14-6.35 | 2.50 | 0.092 | 0.86-7.22 |

Additive Model

| | | | | | | | | | | |
|---|---|---|---|---|---|---|---|---|---|---|
| rs2279343 | A (0.94) | G (0.06) | AA | AG | 2.68 | 0.025 | 1.14-6.35 | 2.50 | 0.092 | 0.86-7.22 |
| | | | | GG | NA | NA | NA | NA | NA | NA |
| rs1038376 | A (0.71) | T (0.29) | AA/AT | AT | 0.79 | 0.622 | 0.31-2.03 | 1.33 | 0.612 | 0.44-4.00 |
| | | | | TT | 4.26 | 0.001 | 1.76-10.27 | 3.56 | 0.011 | 1.33-9.55 |

Recessive model

| | | | | | | | | | | |
|---|---|---|---|---|---|---|---|---|---|---|
| rs1038376 | A (0.71) | T (0.29) | AA/AT | TT | 4.7 | 0.00015 | 2.11-10.46 | 3.21 | 0.01 | 1.32-7.79 |

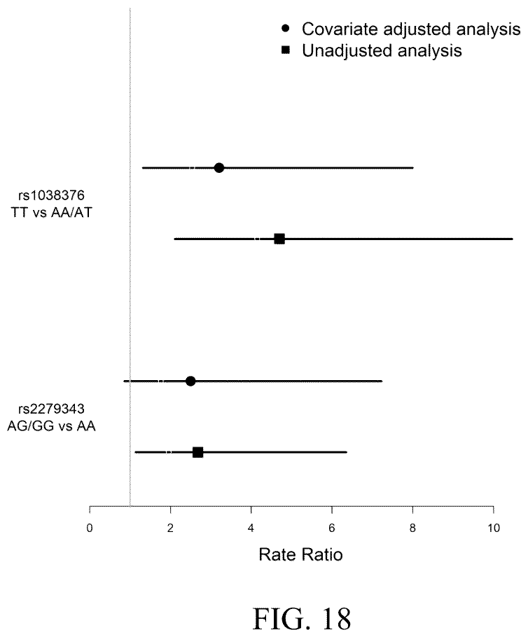

In FIG. 18, the X-axis shows rate ratios of PONV, rate ratio >1 (marked by vertical line) means significantly increased risk of PONV. The rs1038376 variant was associated with significantly greater risk of PONV, both in unadjusted mean (95% CI) 4.7 (2.11-10.46) and covariate adjusted models 3.21(1.32-7.79). CYP2B6 rs2279343 and rs1057868 variants showed greater risk of PONV, but the significance was nominal, on covariate adjustment. The incidence of PONV was up to 4.7 times greater in those with the variant allele (T7) compared to the reference allele (AA/AT) (P=0.00015). On adjusting for covariates, this was reduced to nominal association [rate ratio: 3.21; 95% CI 1.32-7.79 (P=0.01)]. Similarly, AG/GG variants of rs2279343 SNP exhibited up to 2.86 times greater episodes of PONV compared to the wild variant (AA) in the unadjusted models (FIG. 18).

Genetic Associations with Pain Scores (Table 9)

Nominal associations were detected between variants of rs10500282, rs11882424 and rs4803419 and maximum pain scores. As shown in Table 9, those with TT variants of rs4803419 reported lower pain scores compared to the wild type (CC), and those with variants of rs10500282 and rs11882424 reported greater pain scores compared to the referent group.

TABLE 9
CYP2B6 genetic associations with patient reported pain scores, adjusted
for covariates including weight, race, age, surgery type and sex

| | Referent | Minor | | | | | | Covariate adjusted | | |
|---|---|---|---|---|---|---|---|---|---|---|
| | allele | allele | Referent | Alternative | Unadjusted analysis | | | analysis | | |
| SNP | (Freq.) | (Freq.) | Group | group | Beta | P | 95% CI | Beta | P | 95% CI |

Dominant model

| | | | | | | | | | | |
|---|---|---|---|---|---|---|---|---|---|---|
| rs10500282 | T (0.82) | C (0.18) | TT | TC/CC | 1.16 | 0.020 | 0.19-2.13 | 1.18 | 0.024 | 0.16-2.20 |
| rs11882424 | T (0.95) | C (0.05) | TT | TC/CC | 2.19 | 0.007 | 0.63-3.75 | 2.12 | 0.013 | 0.47-3.76 |

Additive Model

| | | | | | | | | | | |
|---|---|---|---|---|---|---|---|---|---|---|
| rs10500282 | T (0.82) | C (0.18) | TT | TC | 1.33 | 0.007 | 0.36-2.29 | 1.31 | 0.010 | 0.33-2.30 |
| | | | | CC | −2.00 | 0.234 | −5.36-1.34 | −2.61 | 0.156 | −6.24-1.03 |
| rs11882424 | T (0.95) | C (0.05) | TT | TC | 2.19 | 0.007 | 0.63-3.75 | 2.12 | 0.013 | 0.47-3.76 |
| | | | | CC | NA | NA | NA | NA | NA | NA |

Recessive model

| | | | | | | | | | | |
|---|---|---|---|---|---|---|---|---|---|---|
| rs4803419 | C (0.65) | T (0.35) | CC/TT | TT | −1.78 | 0.03 | −3.38-−0.19 | −1.70 | 0.049 | −3.39-−0.01 |

CYP2B6 Variants and Methadone Metabolizing Status

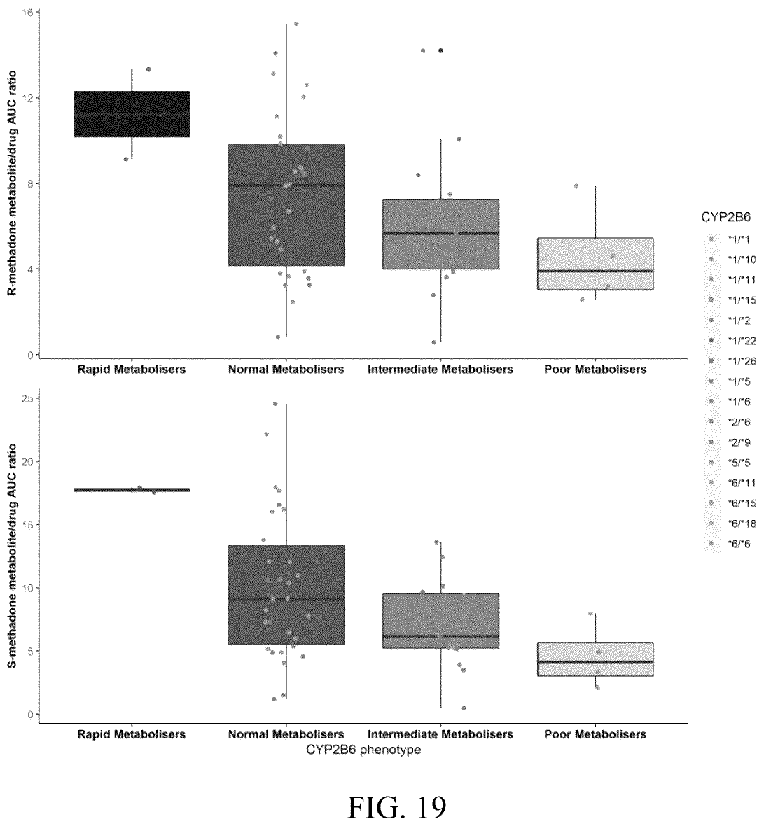

FIG. 19 is a depiction of the phenotype-methadone PK association. The upper graph depicts CYP2B6 phenotype association with metabolite-drug ratio of R-methadone. The lower graph depicts CYP2B6 phenotype association with metabolite-drug ratio of S-methadone. Rapid metabolizers metabolize methadone over 2 times faster than poor metabolizers, for both R- and S enantiomers, and the difference is more pronounced with the S-enantiomer. CYP2B6 poor metabolizers (*6/*6) had >2-fold lower R- and S-methadone metabolites to methadone ratio compared with normal and rapid metabolizers Nominal associations with metabolite/methadone AUC ratio were detected between variants of rs10500282 (p=0.012) and rs1038376 (P=0.032) in unadjusted analysis in additive models. In the co-variate adjusted model, the nominal significance persisted with rs10500282 (p=0.020) and there was a trend with rs1038376 (P=0.079) (Table 10).

TABLE 10
CYP2B6 genetic associations with methadone metabolite to methadone AUC ratio,
adjusted for covariates including weight, race, age, surgery type and sex

| | Referent | Minor | | | | | | Covariate adjusted | | |
|---|---|---|---|---|---|---|---|---|---|---|
| | allele | allele | Referent | Alternative | Unadjusted analysis | | | analysis | | |
| SNP | (Freq.) | (Freq.) | Group | group | Beta | P | 95% CI | Beta | P | 95% CI |

Dominant model

| | | | | | | | | | | |
|---|---|---|---|---|---|---|---|---|---|---|
| rs10500282 | T (0.82) | C (0.18) | AA | AT/TT | −3.99 | 0.012 | −7.05-−0.92 | −3.78 | 0.020 | −6.93-−0.63 |
| rs1038376 | A (0.71) | T (0.29) | TT | TC/CC | −3.29 | 0.032 | −6.27-−0.30 | −2.92 | 0.079 | −6.18-0.35 |

Discussion

Associations between CYP2B6 variants and methadone metabolism and clinical outcomes in perioperative settings discussed above demonstrated that CYP2B6 poor metabolizers (*6/*6) had >2-fold lower R- and S-methadone metabolites to methadone ratio compared with normal and rapid metabolizers in adolescents who received multiple doses of methadone intraoperatively and postoperatively. CYP2B6 rs1038376 variant had significant association with high risk (4.7 fold) for PONV (FIG. 18). In addition, AG/GG variants of rs2279343 SNP had 2.86-fold higher incidence of PONV compared to the wild variant (AA). Maximum postoperative pain scores had nominal associations with CYP2B6 variants, rs10500282, rs11882424 and rs4803419.

Safety and efficacy of the multiple small dose methadone regimen in a similar study cohort, compared to a historical cohort that received non-methadone-based analgesia, is detailed in Example 1. The pharmacokinetics of the same regimen and safe plasma methadone levels (<100 ng/mL) without any clinical respiratory depression even after repeated doses administered every 12 hours have been elucidated. The peak methadone concentration (Cₘₐₓ) was 24.7 (IQR, 19.2-40.8) ng/mL and trough (Cₘᵢₙ) was 4.09 (IQR, 2.74-6.4) ng/mL. The wide range signifies significant inter-person variability in methadone metabolism. Without intending to be bound by any particular theory, this could be the result of polymorphisms in the various enzymes involved in methadone metabolism and also the fluctuating levels of the main binding protein-alpha-acid glycoprotein (AAG).

Methadone undergoes hepatic metabolism, mainly via the CYP enzyme pathway. 10-20% of the parent drug is eliminated in urine. Without intending to be bound by any particular theory, it is believed that methadone undergoes N-demethylation and spontaneous cyclization to form the major metabolites 2-ethylidene-1,5-dimethyl-3,3-diphenylpyrrolidine (EDDP), and 2-ethyl-5-methyl-3,3-diphenylpyrroline (EMDP), both of which have no opioid activity. The primary enzyme involved is the CYP2B6. Other enzymes involved in methadone metabolism include CYP3A4, CYP2D6, CYP2C9 and CYP2C219. Metabolism is stereoselective for R- and S-methadone. CYP2B6 metabolizes S-methadone faster than R-methadone, while CYP2C19 metabolizes S-methadone preferentially than R-methadone.

Most of the pharmacogenetic studies on methadone have been performed in the context of methadone maintenance therapy (MMT) for opioid use disorders. Up to a 17-fold inter-individual variation in blood levels after methadone administration has been demonstrated, and the clearance varies between 0.02-2 L/min. This has contributed to the variation in dose requirements for MMT. No previous study reported associations between CYP2B6 variants and methadone's metabolism and clinical outcomes in perioperative settings, where AAG levels increase due to acute stress and influence methadone's immediate postoperative outcomes. The present Example elucidates methadone's metabolic and clinic outcome variations with CYP2B6 genetic variants in children undergoing major inpatient surgeries.

CYP2B6 is the most significant enzyme mediating stereoselective metabolism of methadone. CYP2B6 expression shows 20- to 250-fold inter-individual variability. Genetic variants in CYP2B6 have been shown to significantly impact methadone PK in healthy adults. The most commonly implicated allele is the *6 haplotype, which is a combination of *4 [rs2279343] and *9 [rs3745274] allelic variants. A prior study involving 64 healthy adult volunteers, and that involved use of a single dose of methadone, demonstrated that CYP2B6*6/*6 was associated with 45% lower clearance in S-methadone and 30% lower clearance in R-methadone compared to the wild type *1/*1. There was a 2.1-fold and 1.7-fold increase in trough and peak concentrations, respectively, of S-methadone in homozygous *6 carriers compared with the non-carriers and a 1.3-fold increase in both trough and peak plasma concentrations of R-methadone. The present Example, which employed multiple doses of methadone in children perioperatively, shows >200% lower methadone metabolite to methadone AUC ratio. Specifically, CYP2B6 phenotypes were compared to methadone metabolism (drug/metabolite ratios), and it was determined that poor metabolizers (*6/*6) had >2-fold lower metabolite/drug AUC ratios, compared with normal (*1/*1) or rapid metabolizers (*1/*22) (FIG. 19). But metabolic differences contributing to variations in clinical outcomes were not observed, which may be due a small number of extreme phenotypes (2 PM and 4 RM) in this Example to detect significant differences in clinical outcomes between phenotypes.

Since CYP2B6's effect on methadone metabolism is stereoselective, influencing (S) methadone greater than (R) enantiomer, the effect on metabolism might not reflect clinical response. Further, other factors like varying levels of AAG, variants on other CYP genes, POR gene could affect PK of methadone. CYP2B6*4 [rs2279343] has previously been reported to be associated with significantly greater methadone metabolism, significant association with methadone dose requirements or response has been reported. In this Example, CYP2B6 variant rs1038376 was found to be significantly associated with greater incidence of PONV after adjusting for multiple comparison, and rs2279343 was found to be nominally associated with greater incidence of PONV.

CYP2B6 polymorphisms have also been found to influence methadone dose requirements for methadone maintenance treatment (“MMT”). Homozygosity for variants rs2279343 (785A>G) and rs3745274 (516G>7) were found to be associated with significantly decreased methadone dosage requirements. Though no significant association with rs3745274 variant was found, rs2279343 was associated with increased risk of PONV. Without intending to be bound by any particular theory, it is believed that the increased risk of PONV may be due to the basic difference in the population groups and the dosages required. Long term methadone management in context of MMT has the potential for autoinduction of CYP2B6, which may not be relevant in postoperative setting. Further, the mean daily dose of methadone in a prior study was 140 mg per day, which is significantly higher than perioperative dosing, especially in the present pediatric Example.

Further genetic ancestry also seems to play a major role in pharmacogenetic associations, as the above earlier studies predominantly comprised Middle Eastern/European ancestry, compared to primarily Caucasian subjects in the present Example. This factor is further highlighted by another study in a Han Chinese population, where rs3745274 variant was associated with increased methadone dose requirements. In the context of analgesic efficacy, when used postoperatively in the present Example, nominal associations of rs10500282, rs11882424 and rs4803419 were found with patient reported maximum postoperative pain scores. Methadone can also cause auto-induction of CYP2B6, which is significant in repeat dosing, though this is less commonly seen with *6 variant. Other minor alleles of CYP2B6 (*5 [rs3211371], *11 [rs35303484]) have been studied, but their effect on methadone disposition and clinical effects remain unclear to date.

In conclusion, associations between CYP2B6 polymorphisms, variations in methadone's metabolism and clinical outcomes of perioperative doses of methadone are disclosed herein. CYP2B6 rs1038376 and rs2279343 were associated with greater risk of methadone induced PONY. CYP2B6 variants of rs10500282, rs11882424 and rs4803419 showed association with maximum pain scores. CYP2B6 variants, rs10500282 and rs1038376 were associated with metabolite/methadone AUC ratio. The present Example advances understanding of the pharmacogenetics affecting perioperative methadone and enables predictive, personalized administration of analgesic therapy to patients. CYP2B6 SNPs, rs1038376 (TT allele) and rs2279343 (AG/GG) had significantly higher risk for methadone-induced PONY. CYP2B6 variants (TC/CC) of rs10500282 and rs11882424 are at risk for higher post-surgical pain scores, and TT variants of rs4803419 reported lower pain scores.

### REFERENCES

Each of the following documents is hereby expressly incorporated by reference herein in its entirety.
1. Gourlay G K, Wilson P R, Glynn C J. Pharmacodynamics and pharmacokinetics of methadone during the perioperative period. Anesthesiology. 1982; 57(6):458-467.
2. Singhal N R, Jones J, Semenova J, et al. Multimodal anesthesia with the addition of methadone is superior to epidural analgesia: A retrospective comparison of intraoperative anesthetic techniques and pain management for 124 pediatric patients undergoing the Nuss procedure. J Pediatr Surg. 2016; 51(4):612-616.
3. Gottschalk A, Durieux M E, Nemergut E C. Intraoperative methadone improves postoperative pain control in patients undergoing complex spine surgery. Anesth Analg. 2011; 112(1):218-223.
4. Machado F C, Vieira J E, de Orange F A, Ashmawi H A. Intraoperative Methadone Reduces Pain and Opioid Consumption in Acute Postoperative Pain: A Systematic Review and Meta-analysis. Anesth Analg. 2019; 129(6):1723-1732.
5. D'Souza R S, Gurrieri C, Johnson R L, Warner N, Wittwer E. Intraoperative methadone administration and postoperative pain control: a systematic review and meta-analysis. PAIN. 2020; 161(2):237-243.
6. Dunn L K, Yerra S, Fang S, et al. Safety profile of intraoperative methadone for analgesia after major spine surgery: An observational study of 1,478 patients. J Opioid Manag. 2018; 14(2):83-87.
7. Ye J, Myung K, Packiasabapathy S, et al. Methadone-based Multimodal Analgesia Provides the Best-in-class Acute Surgical Pain Control and Functional Outcomes With Lower Opioid Use Following Major Posterior Fusion Surgery in Adolescents With Idiopathic Scoliosis. Pediatr Qual Saf. 2020; 5(4):e336.
8. Berde C B, Beyer J E, Bournaki M C, Levin C R, Sethna N F. Comparison of morphine and methadone for prevention of postoperative pain in 3- to 7-year-old children. The Journal of pediatrics. 1991; 119(1 Pt 1):136-141.
9. Sharma A, Tallchief D, Blood J, Kim T, London A, Kharasch E D. Perioperative pharmacokinetics of methadone in adolescents. Anesthesiology. 2011; 115(6):1153-1161.
10. Martin D P, Samora W P, 3rd, Beebe A C, et al. Analgesic effects of methadone and magnesium following posterior spinal fusion for idiopathic scoliosis in adolescents: a randomized controlled trial. J Anesth. 2018; 32(5):702-708.
11. Gornitzky A L, Flynn J M, Muhly W T, Sankar W N. A Rapid Recovery Pathway for Adolescent Idiopathic Scoliosis That Improves Pain Control and Reduces Time to Inpatient Recovery After Posterior Spinal Fusion. Spine Deform. 2016; 4(4):288-295.
12. Stemland C J, Witte J, Colquhoun D A, et al. The pharmacokinetics of methadone in adolescents undergoing posterior spinal fusion. Paediatr Anaesth. 2013; 23(1):51-57.
13. Murphy G S, Szokol J W. Intraoperative Methadone in Surgical Patients: A Review of Clinical Investigations. Anesthesiology. 2019; 131(3):678-692.
14. Kharasch E D. Intraoperative methadone: rediscovery, reappraisal, and reinvigoration? Anesth Analg. 2011; 112(1):13-16.
15. Murphy G S, Avram M J, Greenberg S B, et al. Postoperative Pain and Analgesic Requirements in the First Year after Intraoperative Methadone for Complex Spine and Cardiac Surgery. Anesthesiology. 2020; 132(2):330-342.
16. McCurdy T R, Bhakta V, Eltringham-Smith L J, Gataiance S, Fox-Robichaud A E, Sheffield W P. In vivo clearance of alpha-1 acid glycoprotein is influenced by the extent of its N-linked glycosylation and by its interaction with the vessel wall. J Biomed Biotechnol. 2012; 2012:292730.
17. Israili Z H, Dayton P G. Human alpha-1-glycoprotein and its interactions with drugs. Drug Metab Rev. 2001; 33(2):161-235.
18. Metzger I F, Thomas A E, Evrard C A, et al. Stereoselective Analysis of Methadone and EDDP in Laboring Women and Neonates in Plasma and Dried Blood Spots and Association with Neonatal Abstinence Syndrome. Am J Perinatol. (EFirst).
19. Stumpe M, Miller C, Morton N S, Bell G, Watson D G. High-performance liquid chromatography determination of alphal-acid glycoprotein in small volumes of plasma from neonates. J Chromatogr B Analyt Technol Biomed Life Sci. 2006; 831(1-2):81-84.
20. Centers for Disease Control and Prevention A, G A. Opioid Morphine Equivalent Conversion Factors. 2015.
21. Chidambaran V, Ding L, Moore D L, et al. Predicting the pain continuum after adolescent idiopathic scoliosis surgery: A prospective cohort study. Eur J Pain. 2017; 21(7):1252-1265.
22. Walker L S, Greene J W. The functional disability inventory: measuring a neglected dimension of child health status. J Pediatr Psychol. 1991; 16(1):39-58.
23. Yap Y G, Camm A J. Drug induced Q T prolongation and torsades de pointes. Heart. 2003; 89(11):1363-1372.
24. Tisdale J E, Jaynes H A, Kingery J R, et al. Development and validation of a risk score to predict Q T interval prolongation in hospitalized patients. Circ Cardiovasc Qual Outcomes. 2013; 6(4):479-487.
25. Kuznetsova A, Brockhoff P B, Christensen RHB. lmerTest Package: Tests in Linear Mixed Effects Models. 2017. 2017; 82(13):26.
26. Horst J, Frei-Jones M, Deych E, Shannon W, Kharasch E D. Pharmacokinetics and analgesic effects of methadone in children and adults with sickle cell disease. Pediatric blood & cancer. 2016; 63(12):2123-2130.
27. Israili Z H, Dayton P G. HUMAN ALPHA-1-GLYCOPROTEIN AND ITS INTERACTIONS WITH DRUGS†‡. Drug Metabolism Reviews. 2001; 33(2):161-235.
28. Foster D J, Somogyi A A, Dyer K R, White J M, Bochner F. Steady-state pharmacokinetics of (R)- and (S)-methadone in methadone maintenance patients. Br J Clin Pharmacol. 2000; 50(5):427-440.
29. Lehotay D C, George S, Etter M L, et al. Free and bound enantiomers of methadone and its metabolite, EDDP in methadone maintenance treatment: relationship to dosage? Clin Biochem. 2005; 38(12):1088-1094.
30. Booker P D, Taylor C, Saba G. Perioperative changes in alpha 1-acid glycoprotein concentrations in infants undergoing major surgery. British journal of anaesthesia. 1996; 76(3):365-368.
31. Holley F O, Ponganis K V, Stanski D R. Effects of cardiac surgery with cardiopulmonary bypass on lidocaine disposition. Clin Pharmacol Ther. 1984; 35(5):617-626.
32. Larsson B A, Lonnqvist P A, Olsson G L. Plasma concentrations of bupivacaine in neonates after continuous epidural infusion. Anesthesia and analgesia. 1997; 84(3):501-505.
33. Richlin D M, Reuben S S. Postoperative pain control with methadone following lower abdominal surgery. Journal of clinical anesthesia. 1991; 3(2):112-116.
34. Barratt D T, Coller J K, Hallinan R, et al. ABCB1 haplotype and OPRM1 118A >G genotype interaction in methadone maintenance treatment pharmacogenetics. Pharmgenomics Pers Med. 2012; 5:53-62.
35. Murphy G S, Szokol J W, Avram M J, et al. Intraoperative Methadone for the Prevention of Postoperative Pain: A Randomized, Double-blinded Clinical Trial in Cardiac Surgical Patients. Anesthesiology. 2015; 122(5):1112-1122.
36. Komen H, Brunt L M, Deych E, Blood J, Kharasch E D. Intraoperative Methadone in Same-Day Ambulatory Surgery: A Randomized, Double-Blinded, Dose-Finding Pilot Study. Anesthesia and analgesia. 2019; 128(4):802-810.
37. Alinej ad S, Kazemi T, Zamani N, Hoffman R S, Mehrpour 0. A systematic review of the cardiotoxicity of methadone. EXCLI journal. 2015; 14:577-600.
38. Carlquist J F, Moody D E, Knight S, et al. A Possible Mechanistic Link Between the CYP2C19 Genotype, the Methadone Metabolite Ethylidene-1,5-Dimethyl-3,3-Diphenylpyrrolidene (EDDP), and Methadocahngne-Induced Corrected Q T Interval Prolongation in a Pilot Study. Mol Diagn Ther. 2015; 19(2):131-138.
39. Eap C B, Buclin T, Baumann P. Interindividual Variability of the Clinical Pharmacokinetics of Methadone. Clinical Pharmacokinetics. 2002; 41(14):1153-1193.
40. Chang Y, Fang W B, Lin S N, Moody D E. Stereo-selective metabolism of methadone by human liver microsomes and cDNA-expressed cytochrome P450s: a reconciliation. Basic Clin Pharmacol Toxicol. 2011; 108(1):55-62.
41. Csajka C, Crettol S, Guidi M, Eap C B. Population genetic-based pharmacokinetic modeling of methadone and its relationship with the QTc interval in opioid-dependent patients. Clinical pharmacokinetics. 2016; 55(12):1521-1533.
42. Barratt D T, Coller J K, Hallinan R, et al. ABCB1 haplotype and OPRM1 118A >G genotype interaction in methadone maintenance treatment pharmacogenetics. Pharmgenomics Pers Med. 2012; 5:53-62.
43. Carlquist J F, Moody D E, Knight S, et al. A Possible Mechanistic Link Between the CYP2C19 Genotype, the Methadone Metabolite Ethylidene-1,5-Dimethyl-3,3-Diphenylpyrrolidene (EDDP), and Methadone-Induced Corrected QT Interval Prolongation in a Pilot Study. Mol Diagn Ther. 2015; 19(2):131-138.
44. Romach M K, Piafsky K M, Abel J G, Khouw V, Sellers E M. Methadone binding to orosomucoid (alpha 1-acid glycoprotein): determinant of free fraction in plasma. Clin Pharmacol Ther. 1981; 29(2):211-217.
45. Metzger I F, Thomas A E, Evrard C A, et al. Stereoselective Analysis of Methadone and EDDP in Laboring Women and Neonates in Plasma and Dried Blood Spots and Association with Neonatal Abstinence Syndrome. Am J Perinatol. (EFirst).
46. Stumpe M, Miller C, Morton N S, Bell G, Watson D G. High-performance liquid chromatography determination of alphal-acid glycoprotein in small volumes of plasma from neonates. J Chromatogr B Analyt Technol Biomed Life Sci. 2006; 831(1-2):81-84.
47. Numanagie I, Malikie S, Ford M, et al. Allelic decomposition and exact genotyping of highly polymorphic and structurally variant genes. Nature Communications. 2018; 9(1):828.
48. Lindbom L, Pihlgren P, Jonsson N. PsN-Toolkit—A collection of computer intensive statistical methods for non-linear mixed effect modeling using NONMEM. Computer Methods and Programs in Biomedicine. 2005; 79(3):241-257.
49. R: A Language and Environment for Statistical Computing [computer program]. Vienna, Austria: R Foundation for Statistical Computing; 2021.
50. Jonsson E N, Karlsson M O. Xpose—an S-PLUS based population pharmacokinetic/pharmacodynamic model building aid for NONMEM. Computer Methods and Programs in Biomedicine. 1998; 58(1):51-64.
51. RStudio: Integrated Development Environment for R [computer program]. Boston, M A: RStudio, PBC; 2021.
52. Keizer R J, van Benten M, Beijnen J H, Schellens J H M, Huitema A D R. Piraña and PCluster: A modeling environment and cluster infrastructure for NONMEM. Computer Methods and Programs in Biomedicine. 2011; 101(1):72-79.
53. Beal S L. Ways to Fit a P K Model with Some Data Below the Quantification Limit. Journal of Pharmacokinetics and Pharmacodynamics. 2001; 28(5):481-504.
54. Johansson A M, Karlsson M O. Comparison of methods for handling missing covariate data. AAPS J. 2013; 15(4):1232-1241.
55. Ahmad T, Sabet S, Primerano D A, Richards-Waugh L L, Rankin G O. Tell-tale SNPs: the role of CYP2B6 in methadone fatalities. Journal of analytical toxicology. 2017; 41(4):325-333.
56. Bart G, Lenz S, Straka R J, Brundage R C. Ethnic and genetic factors in methadone pharmacokinetics: a population pharmacokinetic study. Drug Alcohol Depend.
2014; 145:185-193.
57. Crettol S, Deglon J-J, Besson J, et al. ABCB1 and cytochrome P450 genotypes and phenotypes: Influence on methadone plasma levels and response to treatment. Clinical Pharmacology & Therapeutics. 2006; 80(6):668-681.
58. Dennis B B, Bawor M, Thabane L, Sohani Z, Samaan Z. Impact of ABCB1 and CYP2B6 genetic polymorphisms on methadone metabolism, dose and treatment response in patients with opioid addiction: a systematic review and meta-analysis. PloS one. 2014; 9(1).
59. Fonseca Casals F, Fornell T, Diaz L, et al. Contribution of cytochrome P450 and ABCB1 genetic variability on methadone pharmacokinetics, dose requirements, and response. PLoS ONE 2011; 6 (5): e19527. 2011.
60. Kharasch E D, Regina K J, Blood J, Friedel C. Methadone Pharmacogenetics: CYP2B6 Polymorphisms Determine Plasma Concentrations, Clearance, and Metabolism. Anesthesiology. 2015; 123(5):1142-1153.
61. Kringen M K, Chalabianloo F, Bernard J-P, Bramness J G, Molden E, Hoiseth G. Combined Effect of CYP2B6 Genotype and Other Candidate Genes on a Steady-State Serum Concentration of Methadone in Opioid Maintenance Treatment. Therapeutic Drug Monitoring. 2017; 39(5):550-555.
62. Wang S C, Ho I K, Tsou H H, et al. CYP2B6 polymorphisms influence the plasma concentration and clearance of the methadone S-enantiomer. J Clin Psychopharmacol. 2011; 31(4):463-469.
63. Yang H C, Chu S K, Huang C L, et al. Genome-Wide Pharmacogenomic Study on Methadone Maintenance Treatment Identifies SNP rs17180299 and Multiple Haplotypes on CYP2B6, SPON1, and GSG1L Associated with Plasma Concentrations of Methadone R- and S-enantiomers in Heroin-Dependent Patients. PLoS Genet. 2016; 12(3):e1005910.
64. Sinha J, Al-Sallami H S, Duffull S B. Choosing the Allometric Exponent in Covariate Model Building. Clin Pharmacokinet. 2019; 58(1):89-100.
65. Booker P D, Taylor C, Saba G. Perioperative changes in alpha 1-acid glycoprotein concentrations in infants undergoing major surgery. Br J Anaesth. 1996; 76(3):365-368.
66. Larsson B A, Lonnqvist P A, Olsson G L. Plasma concentrations of bupivacaine in neonates after continuous epidural infusion. Anesth Analg. 1997; 84(3):501-505.
67. Eap C B, Cuendet C, Baumann P. Binding of d-methadone, 1-methadone, and dl-methadone to proteins in plasma of healthy volunteers: role of the variants of alpha 1-acid glycoprotein. Clin Pharmacol Ther. 1990; 47(3):338-346.
68. Jiang H H, Liu J, Wang Y C, et al. The Impact of Gene Polymorphisms on Anticoagulation Control With Warfarin. Clin Appl Thromb Hemost. 2018; 24(4):640-646.
69. Chen W Q, Shu Y, Li Q, et al. Polymorphism of ORM1 is associated with the pharmacokinetics of telmisartan. PLoS One. 2013; 8(8):e70341.
70. Gerber J G, Rhodes R J, Gal J. Stereoselective metabolism of methadone N-demethylation by cytochrome P4502B6 and 2C19. Chirality. 2004; 16(1):36-44.
71. Lehotay D C, George S, Etter M L, et al. Free and bound enantiomers of methadone and its metabolite, EDDP in methadone maintenance treatment: relationship to dosage? Clin Biochem. 2005; 38(12):1088-1094.
72. Crews K R, Monte A A, Huddart R, et al. Clinical Pharmacogenetics Implementation Consortium Guideline for CYP2D6, OPRM1, and COMT Genotypes and Select Opioid Therapy. Clin Pharmacol Ther. 2021.
73. Lima J J, Thomas C D, Barbarino J, et al. Clinical Pharmacogenetics Implementation Consortium (CPIC) Guideline for CYP2C19 and Proton Pump Inhibitor Dosing. Clin Pharmacol Ther. 2020.
74. Birdwell K A, Decker B, Barbarino J M, et al. Clinical Pharmacogenetics Implementation Consortium (CPIC) Guidelines for CYP3A5 Genotype and Tacrolimus Dosing. Clin Pharmacol Ther. 2015; 98(1):19-24.
75. Packiasabapathy S, Aruldhas B W, Horn N, et al. Pharmacogenomics of methadone: a narrative review of the literature. Pharmacogenomics. 2020; 21(12):871-887.
76. Richards-Waugh L L, Primerano D A, Dementieva Y, Kraner J C, Rankin G O. Fatal methadone toxicity: potential role of CYP3A4 genetic polymorphism. J Anal Toxicol. 2014; 38(8):541-547.
77. Thorn C F, Lamba J K, Lamba V, Klein T E, Altman R B: PharmGKB summary: very important pharmacogene information for CYP2B6. Pharmacogenet Genomics 2010, 20:520-523.
78. Tzvetkov M V: OCT1 pharmacogenetics in pain management: is a clinical application within reach? Pharmacogenomics 2017, 18:1515-1523.
79. Crettol S, Deglon J-J, Besson J, Croquette-Krokar M, Hammig R, Gothuey I, Monnat M, Eap C B: ABCB1 and cytochrome P450 genotypes and phenotypes: Influence on methadone plasma levels and response to treatment. Clin Pharmacol Ther 2006, 80:668-681.
80. Carroll J L, Loughlin G M: Diagnostic criteria for obstructive sleep apnea syndrome in children. Pediatr Pulmonol 1992, 14:71-74.
81. Ye J, Myung K, Packiasabapathy S, Yu J S, Jacobson J E, Whittaker S C, Castelluccio P, Drayton Jackson M, Sadhasivam S: Methadone-based Multimodal Analgesia Provides the Best-in-class Acute Surgical Pain Control and Functional Outcomes With Lower Opioid Use Following Major Posterior Fusion Surgery in Adolescents With Idiopathic Scoliosis. Pediatr Qual Saf 2020, 5:e336.
82. Sadhasivam S, Aruldhas B W, Packiasabapathy S, Overholser B R, Zhang P, Zang Y, Renschler J S, Fitzgerald R E, Quinney S K: A Novel Perioperative Multidose Methadone-Based Multimodal Analgesic Strategy in Children Achieved Safe and Low Analgesic Blood Methadone Levels Enabling Opioid-Sparing Sustained Analgesia With Minimal Adverse Effects. Anesth Analg 2021.
83. Brodin K, Lybeck A, Beck O, Liljeberg P, Lindstrom U, Borg S, Hjemdahl P: 175 MONITORING O F METHADONE DOSE INTAKE: IMPORTANCE O F URINARY pH. Ther Drug Monit 1997, 19:591.
84. Totah R A, Sheffels P, Roberts T, Whittington D, Thummel K, Kharasch E D: Role of CYP2B6 in stereoselective human methadone metabolism. Anesthesiology 2008 , 108:363-374.
85. Gerber J G, Rhodes R J, Gal J: Stereoselective metabolism of methadone N-demethylation by cytochrome P4502B6 and 2C19. Chirality 2004, 16:36-44.
86. Chang Y, Fang W B, Lin S N, Moody D E: Stereo-selective metabolism of methadone by human liver microsomes and cDNA-expressed cytochrome P450s: a reconciliation. Basic Clin Pharmacol Toxicol 2011, 108:55-62.
87. Totah R A, Allen K E, Sheffels P, Whittington D, Kharasch E D: Enantiomeric metabolic interactions and stereoselective human methadone metabolism. J Pharmacol Exp Ther 2007, 321:389-399.
88. Nixon G M, Kermack A S, McGregor C D, Davis G M, Manoukian J J, Brown K A, Brouillette R T: Sleep and breathing on the first night after adenotonsillectomy for obstructive sleep apnea. Pediatr Pulmonol 2005, 39:332-338.
89. Levran O, Peles E, Randesi M, Shu X, Ott J, Shen P H, Adelson M, Kreek M J: Association of genetic variation in pharmacodynamic factors with methadone dose required for effective treatment of opioid addiction. Pharmacogenomics 2013, 14:755-768.
90. Levran O, Peles E, Hamon S, Randesi M, Adelson M, Kreek M J: CYP2B6 SNPs are associated with methadone dose required for effective treatment of opioid addiction. Addict Biol 2013, 18:709-716.
91. Kharasch E D, Regina K J, Blood J, Friedel C: Methadone pharmacogenetics: CYP2B6 polymorphisms determine plasma concentrations, clearance and metabolism. Anesthesiology 2015, 123:1142-1153.
92. Crettol S, Deglon J J, Besson J, Croquette-Krokkar M, Gothuey I, Hammig R, Monnat M, Huttemann H, Baumann P, Eap C B: Methadone enantiomer plasma levels, CYP2B6, CYP2C19, and CYP2C9 genotypes, and response to treatment. Clin Pharmacol Ther 2005 , 78:593-604.
93. Uehlinger C, Crettol S, Chassot P, Brocard M, Koeb L, Brawand-Amey M, Eap CB: Increased (R)-Methadone Plasma Concentrations by Quetiapine in Cytochrome P450s and ABCB1 Genotyped Patients. J Clin Psychopharmacol 2007, 27:273-278.
94. Fonseca Casals F, Fornell T, Diaz L, Pastor A, Cuyas E, Pizarro Lozano M, Khymenets O, Farre Albaladejo M, Torrens M: Contribution of cytochrome P450 and ABCB1 genetic variability on methadone pharmacokinetics, dose requirements, and response. PLoS ONE 2011; 6 (5): e19527 2011.
95. Oneda B, Crettol S, Jaquenoud Sirot E, Bochud M, Ansermot N, Eap C B: The P450 oxidoreductase genotype is associated with CYP3A activity in vivo as measured by the midazolam phenotyping test. Pharmacogenet Genomics 2009, 19:877-883.
96. Coller J K, Barratt D T, Dahlen K, Loennechen M R, Somogyi A A: ABCB1 genetic variability and methadone dosage requirements in opioid-dependent individuals. Clin Pharmacol Ther 2006, 80:682-690.
97. Hung C C, Chiou M R, Huang B H, Hsieh Y W, Hsieh T J, Huang C L, Lane H Y: Impact of genetic polymorphisms in ABCB1, CYP2B6, OPRM1, ANKK1 and DRD2 genes on methadone therapy in Han Chinese patients. Pharmacogenomics 2011, 12:1525-1533.
98. Walter C, Lotsch J: Meta-analysis of the relevance of the OPRM1 118A>G genetic variant for pain treatment. Pain 2009, 146:270-275.
99. Lotsch J, Skarke C, Wieting J, Oertel B G, Schmidt H, Brockmoller J, Geisslinger G: Modulation of the central nervous effects of levomethadone by genetic polymorphisms potentially affecting its metabolism, distribution, and drug action. Clin Pharmacol Ther 2006, 79:72-89.
100. Dennis B B, Bawor M, Thabane L, Sohani Z, Samaan Z: Impact of ABCB1 and CYP2B6 genetic polymorphisms on methadone metabolism, dose and treatment response in patients with opioid addiction: a systematic review and meta-analysis. PLoS One 2014, 9.
101. Dobrinas M, Crettol S, Oneda B, Lahyani R, Rotger M, Choong E, Lubomirov R, Csajka C, Eap C B: Contribution of CYP2B6 alleles in explaining extreme (S)-methadone plasma levels: a CYP2B6 gene resequencing study. Pharmacogenet Genomics 2013 , 23:84-93.
102. Csajka C, Crettol S, Guidi M, Eap C B: Population genetic-based pharmacokinetic modeling of methadone and its relationship with the QTc interval in opioid-dependent patients. Clin Pharmacokinet 2016, 55:1521-1533.
103. Sadhasivam S, Chidambaran V, Zhang X, Meller J, Esslinger H, Zhang K, Martin L J, McAuliffe J: Opioid-induced respiratory depression: ABCB1 transporter pharmacogenetics. Pharmacogenomics J 2015, 15:119-126.
104. Chidambaran V, Mavi J, Esslinger H, Pilipenko V, Martin L J, Zhang K, Sadhasivam S: Association of OPRM1 A118G variant with risk of morphine-induced respiratory depression following spine fusion in adolescents. Pharmacogenomics J 2015, 15:255-262.

1. A method for controlling pain in a patient in need thereof, the method comprising administering to the patient multiple low doses of methadone, wherein each subsequent dose of methadone is administered within an effective period of time from a previously administered dose of methadone, and wherein the patient has no respiratory depression and no QT prolongation.

2. (canceled)

3. The method of claim 1, further comprising administering to the patient at least one additional active ingredient.

4. (canceled)

5. The method of claim 1, wherein the effective period of time is about 12 hours.

6. (canceled)

7. The method of claim 1, wherein the pain is associated with a surgery performed or to be performed on the patient.

8. (canceled)

9. The method of claim 7, wherein the methadone is administered to the patient preoperatively.

10. The method of claim 7, wherein the methadone is administered to the patient intraoperatively or postoperatively or both intraoperatively and postoperatively.

11. (canceled)

12. The method of claim 7, wherein the patient has a lower incidence of chronic pain after the surgery relative to an incidence of chronic pain in a population that does not receive multiple small doses of methadone.

13. The method of claim 7, wherein the patient has an improved postoperative functional disability index score relative to a postoperative functional disability index score in a population that does not receive multiple small doses of methadone.

14. (canceled)

15. The method of claim 7, wherein a concentration of postoperative alpha-1 acid glycoprotein (AAG) in the patient changes after surgery and influences a response and a pain relief profile of the methadone.

16. (canceled)

17. The method of claim 1, wherein each dose of methadone is at a dose of 0.01 mg/kg of weight of the patient to 1 mg/kg of weight of the patient.

18. (canceled)

19. (canceled)

20. (canceled)

21. The method of claim 1, wherein the multiple low doses of methadone comprise 3 or 4 low doses of methadone.

22. (canceled)

23. (canceled)

24. (canceled)

25. A method for providing personalized analgesic therapy to a surgical patient to improve post-surgical outcomes, comprising:
directing preoperative genotyping of a patient in need of surgery to determine what allele is present at one or more gene loci to obtain a patient-specific genetic data set, wherein the one or more gene loci is selected from the group consisting of a locus that encodes an enzyme, a receptor or other protein associated with methadone metabolism or response, a locus that encodes AAG and combinations thereof;
producing a prediction of the patient's response to perioperative methadone administration based on the data set; and
making a positive or negative determination whether to administer methadone perioperatively to the patient based on the prediction.

26. The method of claim 25, further comprising determining a precision dosing of methadone for the patient based on the genetic data set.

27. (canceled)

28. The method of claim 26 wherein the method further comprises, after said determining, directing administration of methadone to the patient.

29. The method of claim 25 wherein the administration of methadone to the patient comprises administration of multiple low doses of methadone, wherein each subsequent dose of methadone is administered within an effective period of time from a previously administered dose of methadone.

30. (canceled)

31. The method of claim 28 further comprising administering to the patient at least one additional active ingredient.

32. (canceled)

33. The method of claim 29 wherein the effective period of time is about 12 hours.

34. The method of claim 29 wherein the patient's pain is controlled for about 12 hours.

35. The method of claim 29 wherein each dose of methadone is a dose of 0.01 mg/kg of weight of the patient to 1 mg/kg of weight of the patient.

36. (canceled)

37. (canceled)

38. (canceled)

39. (canceled)

40. (canceled)

41. (canceled)

42. The method of claim 25 wherein the genotyping comprises determining the allele present at a gene locus that encodes an enzyme selected from the group consisting of CYP2B6, CYP2D6, CYP2C19, CYP2C9, CYP3A4, ORM1, ABCB1, FAAH, OPRM1 and combinations thereof.

43. (canceled)

44. (canceled)

45. The method of claim 25 wherein the genotyping comprises determining the allele present at a gene locus that encodes a member selected from the group consisting of ORM1, ORM2 and combinations thereof.

46. The method of claim 25 wherein the prediction comprises a prediction whether the patient, if administered methadone, will experience an acceptable analgesic effect, nausea, vomiting, excessive sedation, or any combination thereof.

47. (canceled)

48. (canceled)

49. The method of claim 43 wherein the allele of CYP2B6 predicts the clearance of an R-methadone and an S-methadone enantiomer.

50. The method of claim 42 wherein the genotyping comprises determining the allele present at a gene locus that encodes intronic CYP3A4 SNP rs2246709 .

51. The method of claim 45 wherein changing the patient's perioperative plasma concentrations of AAG and the SNP of AAG, ORM1 SNP rs17650 independently increases the volume of distribution of an R-methadone enantiomer and an S-methadone enantiomer, thereby aiding an optimal and precision dosing of methadone.

52. The method of claim 25 wherein the genotyping comprises determining the allele present at a gene locus that encodes OPRM1, wherein the prediction is that a patient with an OPRM1 rs1799971 GG or GA genotype will require 20% higher average in-hospital opioid use than AA genotype, and a patient with AA genotype will have a higher incidence of PONV and a longer hospital stay relative to a patient without AA genotype.

53. The method of claim 42 wherein a patient with a variant in ABCB1 is determined to have significantly higher risk for PONY than a patient without ABCB1.

54. The method of claim 53 wherein the variant is selected from the group consisting of rs1128503 and rs1045642.

55. The method of claim 42 wherein a patient with a homologous/heterologous variant of FAAH SNPs is expected to have 15-20% lower in-hospital opioid use compared to a patient with wild-type.

56. The method of claim 55 wherein the variant is selected from the group consisting of rs3766246 and rs4141964.

57. The method of claim 42 wherein a patient with a wild type variant of FAAH rs3766246 is expected to have a higher incidence of PONV and one-day longer hospital stay after surgery than a patient without the wild type variant of FAAH.

58. The method of claim 25 wherein the genotyping comprises determining the allele present at a gene locus that encodes OPRM1, and wherein a patient with POR rs1057868 TT or CT genotype is expected to have higher incidence of PONV than a patient with wild type, CC.
